(19) United States
(12) Patent Application Publication
(10) Pub. No.: US 2021/0008209 A1
(43) Pub. Date: Jan. 14, 2021

(54) COMBINATION THERAPY FOR TTR AMYLOIDOSIS

(71) Applicant: Corino Therapeutics, Inc., Mew York, NY (US)

(72) Inventor: Michael J. Roberts, Charlotte, NC (US)

(21) Appl. No.: 16/980,631

(22) PCT Filed: Mar. 12, 2019
(86) PCT No.: PCT/US19/21812

§ 371 Date: Sep. 14, 2020

Related U.S. Application Data
(60) Provisional application No. 62/641,747, filed on Mar. 12, 2018.

(51) Int. Cl. A61K 45/06; A61K 31/12; C12N 15/11

(52) U.S. Cl. CPC A61K 45/06; C12N 15/11; A61K 31/12

(57) ABSTRACT

The present invention is directed to compositions and methods for the treatment of transthyretin-associated (TTR) amyloidosis and in particular, compositions and methods that employ an effective amount of tolcapone and an RNAi molecule in combination for the treatment of transthyretin-associated amyloidosis.

### CROSS-REFERENCE TO RELATED APPLICATION

This application claims priority to U.S. Provisional Patent Application No. 62/641,747, filed Mar. 12, 2018, the contents of which is incorporated by reference in its entirety.

### FIELD OF THE INVENTION

The present invention is directed to compositions and methods for the treatment of transthyretin-associated (TTR) amyloidosis and in particular, compositions and methods that employ an effective amount of tolcapone and at least one RNAi molecule in combination for the treatment of transthyretin-associated amyloidosis.

### BACKGROUND OF THE INVENTION

Transthyretin (TTR) protein is a serum and cerebrospinal fluid carrier of the thyroid hormone thyroxine and retinol. Mutations in the TTR gene can result in a destabilization of the TTR protein, leading to abnormal aggregation and amyloidosis, i.e., the buildup of amyloid proteins in various tissues and organs. Specifically, the destabilization is understood to result from dissociation of the homotetrameric protein into aggregation-prone monomers.

More than 130 amyloidogenic TTR gene mutations have been identified. The vast majority of these mutations are located at exon 2 to 4. The most common (and geographically distributed) mutation involves substitution of methionine for valine at position 30 (Val30Met). Extracellular deposition of wild-type and/or variant TTR aggregates in the heart, lung, gastrointestinal tract, and peripheral nerves is associated with wild-type ATTR-cardiomyopathy (senile systemic amyloidosis (SSA)), hereditary ATTR-polyneuropathy (familial amyloidotic polyneuropathy (FAP)), hereditary ATTR-cardiomyopathy (familial amyloid cardiomyopathy (FAC)), and more rarely, hereditary ATTR-leptomeningeal (central nervous system amyloidosis (CNSA)).

Despite the considerable effort that has been made in the field, there remains a need for effective approaches to the treatment of TTR-associated amyloidosis (ATTR).

### SUMMARY OF THE INVENTION

The invention directed to compositions and methods for treatment of transthyretin-associated amyloidosis.

In a first aspect, the invention provides a composition comprising (i) an effective amount of tolcapone and (ii) an effective amount of at least one RNAi molecule.

In one embodiment, the RNAi molecule is selected from the group consisting of siRNA, miRNA, shRNA and combinations thereof.

In a particular embodiment, the RNAi molecule is a double-stranded siRNA, wherein (a) each strand of the siRNA molecule is about 18 to about 22 ribonucleotides in length; and (b) one strand of the siRNA molecule comprises a ribonucleotide sequence that is substantially complimentary to an mRNA sequence encoding SEQ ID. No.: 1 or a fragment thereof

In a particular embodiment, the RNAi molecule is a double-stranded siRNA, wherein (a) each strand of the siRNA molecule is about 18 to about 22 ribonucleotides in length; and (b) one strand of the siRNA molecule comprises a ribonucleotide sequence that is fully complimentary to an mRNA sequence encoding SEQ ID. No.: 1.

In one embodiment, the RNAi molecule is a blunt 18-mer, a blunt 19-mer, a blunt 21-mer, a blunt 23-mer, a blunt 25-mer or a blunt or 27-mer.

In one embodiment, the RNAi molecule contains at least one nucleotide overhang. In a particular embodiment, the overhang is a two-nucleotide (2-nt) or 3-nucleotide (3-nt) overhang at the 3′ end of the RNAi molecule, the 5′ end of the RNAi molecule or both the 3′ and 5′ ends of the RNAi molecule. In one embodiment, the nucleotide overhang comprises one or more non-ribonucleotides.

In one embodiment, the RNA molecule comprises at least one chemical modification selected from the group consisting of sugar modifications, base modifications, terminal modifications, backbone modifications or combinations thereof.

In a particular embodiment, the RNAi molecule comprises at least one sugar modification and more particularly, a modification to the ribose ring or a ribose ring substituent group, or replacement of the sugar moiety with a non-sugar moiety.

In a second aspect, the invention provides a pharmaceutical composition comprising (i) an effective amount of tolcapone; (ii) an effective amount of at least one siRNAi molecule and (iii) at least one pharmaceutical carrier.

In one embodiment, the siRNA molecule is conjugated to the at least one pharmaceutical carrier. In another embodiment, the siRNA is encapsulated by or non-covalently associated with the at least one pharmaceutical carrier.

In a particular embodiment, the at least one pharmaceutical carrier is selected from the group consisting of carbohydrates, lipids, peptides, protein, nucleic acid molecules, synthetic polymers or combinations thereof.

In one embodiment, the pharmaceutical carrier is a nanocarrier. In a particular embodiment, the nanocarrier comprises cationic lipids, cationic polymers, cationic peptides or combinations thereof.

In a particular embodiment, the invention provides a lipoplex comprising a positively-charged cationic lipid and an RNAi molecule comprises a ribonucleotide sequence that is at least substantially complimentary to an mRNA sequence encoding SEQ ID. No.: 1.

In another particular embodiment, the invention provides a polyplex comprising a cationic polymer and an RNAi molecule comprises a ribonucleotide sequence that is substantially complimentary to an mRNA sequence encoding SEQ ID. No.: 1.

In certain embodiments, the pharmaceutical composition is targeted to a particular tissue, cell type, cellular compartment or combination thereof. In a particular embodiment, the pharmaceutical composition is targeted to the liver.

In a third aspect, the invention provides a method of treating transthyretin-associated amyloidosis in a subject in need thereof comprising co-administering (i) an effective amount of tolcapone and (ii) an effective amount of at least one RNAi molecule, thereby treating the transthyretin-associated amyloidosis

In a particular embodiment, the transthyretin-associated amyloidosis is hereditary ATTR-polyneuropathy (familial amyloid polyneuropathy (FAP)) or hereditary ATTR-cardiomyopathy (familiar amyloid cardiomyopathy (FAC)) or a mixture of disease manifestations.

In one embodiment, the form of co-administration is selected from the group consisting of simultaneous administration, sequential administration, overlapping administration, interval administration, continuous administration, or a combination thereof.

In one embodiment, the tolcapone and the RNAi molecule have different dosing schedules.

In a particular embodiment, the tolcapone and the RNAi molecule are co-administered systemically. In one embodiment, the tolcapone is administered orally and the RNAi molecule is administered by intravenous injection.

In a particular embodiment, the tolcapone and the RNAi molecule are co-administered in different unit dosage forms. In one embodiment, tolcapone is administered as a solid unit dosage form (e.g., a tablet or capsule) and the RNAi molecule is administered as a liquid unit dosage form (e.g., an intravenous or subcutaneous injection).

In one embodiment, the tolcapone and the RNAi molecule are co-administered in a single dose. In another embodiment, the tolcapone and the RNAi molecule are co-administered in multiple doses.

In one embodiment, the co-administration produces a synergistic effect, i.e., a therapeutic affect that is greater than the sum of the therapeutic effects of the individual components of the combination. In another embodiment, the co-administration produces an additive effect.

### DETAILED DESCRIPTION OF THE INVENTION

### Definitions

“G,” “C,” “A,” “T” and “U” each generally stand for a nucleotide that contains guanine, cytosine, adenine, thymidine and uracil as a base, respectively. “T” and “dT” are used interchangeably herein and refer to a deoxyribonucleotide wherein the nucleobase is thymine, e.g., deoxyribothymine. However, it will be understood that the term “ribonucleotide” or “nucleotide” can also refer to a modified nucleotide, as further detailed below, or a surrogate replacement moiety. Guanine, cytosine, adenine, and uracil may be replaced by other moieties without substantially altering the base pairing properties of an oligonucleotide comprising a nucleotide bearing such replacement moiety. For example, without limitation, a nucleotide comprising inosine as its base may base pair with nucleotides containing adenine, cytosine, or uracil. Hence, nucleotides containing uracil, guanine, or adenine may be replaced in the nucleotide sequences of dsRNA featured in the invention by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured in the invention.

The term “aadministering” as used herein refers to providing a therapeutic agent(s) to a subject and includes, but is not limited to, administering by a medical professional and self-administering.

The term “anti-amyloid agent” as used herein refers to an agent which is capable of producing an immune response against an amyloid plaque component in a vertebrate subject, when administered by active or passive immunization techniques.

The term “antisense strand” as used herein refers to the strand of a dsRNA which includes a region that is at least substantially complementary to the target sequence. As used herein, the term “region of complementarity” refers to the region on the antisense strand that is at least substantially complementary to a sequence, for example a target sequence. Where the region of complementarity is not fully complementary to the target sequence, the mismatches are typically in terminal regions and, if present, are generally in a terminal region or regions, e.g., within 6, 5, 4, 3, or 2 nucleotides of the 5′ and/or 3′ terminus.

The term “attenuating expression” as used herein with reference to the TTR gene or mRNA encoding the TTR protein means administering or expressing an amount of interfering RNA (e.g., an siRNA) to reduce translation of the TTR mRNA into the TTR protein, either through mRNA cleavage or through direct inhibition of translation. The TTR gene may be wild-type or mutant. The terms “inhibit,” “silencing,” and “attenuating” as used herein refer to a measurable reduction in expression of the TTR mRNA or the corresponding protein as compared with the expression of the TTR mRNA or the corresponding TTR protein in the absence of an interfering RNA. The reduction in expression of the TTR mRNA or the corresponding protein is commonly referred to as “knock-down” and is reported relative to levels present following administration or expression of a non-targeting control RNA (e.g., a non-targeting control siRNA). Knock-down of expression of an amount including and between 50% and 100% is contemplated by embodiments herein. However, it is not necessary that such knock-down levels be achieved for purposes of the present invention. Attenuating expression of the TTR by an RNAi molecule can be inferred in a human or other mammal by observing an improvement in symptoms TTR associated amyloidosis.

The term “blunt” or “blunt ended” as used herein in reference to a dsRNA means that there are no unpaired nucleotides or nucleotide analogs at a given terminal end of a dsRNA, i.e., no nucleotide overhang. One or both ends of a dsRNA can be blunt. Where both ends of a dsRNA are blunt, the dsRNA is said to be blunt ended. To be clear, a “blunt ended” dsRNA is a dsRNA that is blunt at both ends, i.e., no nucleotide overhang at either end of the molecule. Most often such a molecule will be double-stranded over its entire length.

The term “catechol-O-methyltransferase inhibitor” or “COMT inhibitor” refers to compounds that inhibit the action of catechol-O-methyl transferase, an enzyme that is involved in degrading neurotransmitters (Mannisto and Kaakkola, Pharm. Rev., 1999, vol 51, p. 593-628). COMT inhibitor activity can be determined by methods known in the art, for instance the method disclosed in Zürcher et al (Biomedical Chromatography, 1996, vol. 10, p. 32-36). Several COMT inhibitors have been described. Tolcapone, entacapone, and nitecapone belong to the so called “second generation COMT inhibitors”, which have been shown to be potent, highly selective, and orally active COMT inhibitors. Nitrocatechol is the key structure in these molecules (Pharm. Rev., 1999, vol 51, p. 593-628, supra).

The term “chemical modification” as used herein refers to any modification of the chemical structure of the nucleotides that differs from nucleotides of native RNA or RNAi molecules. The modification may achieve any intended result, including an increase in affinity and/or nuclease resistance. In certain embodiments, the term “chemical modification” can refer to certain forms of RNA that are naturally occurring in certain biological systems, for example 2′-O-methyl modifications or inosine modifications.

The term “coding region” refers to that portion of the TTR gene or mRNA which either naturally or normally codes for the expression product of the gene in its natural genomic environment, i.e., the region coding in vivo for the native expression product of the gene, i.e., the TTR protein. The coding region of the gene (and the coding region of mRNA) beings with a start codon: ATG in DNA and AUG in mRNA code for the amino acid methionine, Met. The coding region of a gene always ends with a stop codon TAA, TAG, or TGA (in mRNA, these have a U instead of a T). The sequence between start and stop codons contains nucleotides in multiples of three, encoding the sequence of amino acids in the protein. The untranslated regulatory regions (denoted by UTR) include a site for the ribosome to bind before the start codon (5′ UTR) and a region after the stop codon (3′ UTR).

The term “co-administration” as used herein refers to administration of two or more therapeutic agents (e.g., a TTR kinetic stabilizer and a TTR genetic silencer) together in a coordinated fashion. For example, the co-administration can be simultaneous administration, sequential administration, overlapping administration, interval administration, continuous administration, or a combination thereof.

The term “complimentary” as used herein refers to the ability of polynucleotides to form base pairs with one another. Base pairs are typically formed by hydrogen bonds between nucleotide units in antiparallel polynucleotide strands. Complementary polynucleotide strands can base pair in the Watson-Crick manner (e.g., A to T, A to U, C to G), or in any other manner that allows for the formation of duplexes. When using RNA as opposed to DNA, uracil rather than thymine is the base that is considered to be complementary to adenosine. However, when a U is denoted in the context of the present invention, the ability to substitute a T is implied, unless otherwise stated. Perfect complementarity or 100% complementarity refers to the situation in which each nucleotide unit of one polynucleotide strand can hydrogen bond with a nucleotide unit of a second polynucleotide strand. Less than perfect complementarity refers to the situation in which some, but not all, nucleotide units of two strands can hydrogen bond with each other. For example, for two 20-mers, if only two base pairs on each strand can hydrogen bond with each other, the polynucleotide strands exhibit 10% complementarity. In the same example, if 18 base pairs on each strand can hydrogen bond with each other, the polynucleotide strands exhibit 90% complementarity. As used herein, “substantially complementary” means that in a hybridized pair of nucleobase or nucleotide sequence molecules, at least 85%, but not all, of the bases in a contiguous sequence of a first oligonucleotide will hybridize with the same number of bases in a contiguous sequence of a second oligonucleotide. The contiguous sequence may comprise all or a part of a first or second nucleotide sequence. As used herein, “partially complementary” means that in a hybridized pair of nucleobase or nucleotide sequence molecules, at least 70%, but not all, of the bases in a contiguous sequence of a first oligonucleotide will hybridize with the same number of bases in a contiguous sequence of a second oligonucleotide. The contiguous sequence may comprise all or a part of a first or second nucleotide sequence.

The term “double-stranded RNA” or “dsRNA,” as used herein, refers to a complex of ribonucleic acid molecules, having a duplex structure comprising two anti-parallel and substantially complementary, as defined above, nucleic acid strands. In general, the majority of nucleotides of each strand are ribonucleotides, but optionally, each or both strands can also include at least one non-ribonucleotide, e.g., a deoxyribonucleotide and/or a modified nucleotide. In addition, as used herein, “dsRNA” may include chemical modifications to ribonucleotides, including substantial modifications at multiple nucleotides and including all types of modifications disclosed herein or known in the art. A dsRNA molecule need not be completely double-stranded, but comprises at least one double-stranded region comprising at least one functional double-stranded silencing element.

The term “effective amount” as used herein means the amount of a compound that, when administered to a mammal or other subject for treating a disease, is sufficient to effect such prophylaxis or treatment for the disease. The “therapeutically effective amount” or “prophylactically effective amount” will vary depending on the compound, the disease and its severity and the age, weight, etc., of the subject.

The term “equivalent” as used herein with reference to an amino acid residue refers to an amino acid capable of replacing another amino acid residue in a polypeptide without substantially altering the structure and/or functionality of the polypeptide. Equivalent amino acids thus have similar properties such as bulkiness of the side-chain, side chain polarity (polar or non-polar), hydrophobicity (hydrophobic or hydrophilic), pH (acidic, neutral or basic) and side chain organization of carbon molecules (aromatic/aliphatic). As such, “equivalent amino acid residues” can be regarded as “conservative amino acid substitutions”.

The term “expression vector” as used herein refers to a nucleic acid molecule encoding a gene that is expressed in a host cell. Typically, an expression vector comprises a transcription promoter, a gene, and a transcription terminator. Gene expression is usually placed under the control of a promoter, and such a gene is said to be “operably linked to” the promoter. Similarly, a regulatory element and a core promoter are operably linked if the regulatory element modulates the activity of the core promoter. Simpler vectors called “transcription vectors” are only capable of being transcribed but not translated: they can be replicated in a target cell but not expressed, unlike expression vectors. Transcription vectors are used to amplify their insert.

The term “gene silencer” as used herein refers to an attenuation of gene expression, and more particularly, a measurable reduction in expression of the corresponding mRNA or protein as compared with the expression of the corresponding mRNA or protein in the absence of an interfering RNA.

The term “guide strand” as used herein refers to a single stranded nucleic acid molecule of a dsRNAi molecule which has a sequence sufficiently complementary to that of a target RNA to result in RNA interference. In some embodiments, Dicer cleavage is not required for the incorporation of a guide strand into RISC. In some embodiments after cleavage of the dsRNAi molecule by Dicer, a fragment of the guide strand remains associated with RISC, binds a target RNA as a component of the RISC complex, and promotes cleavage of a target RNA by RISC. A guide strand is an antisense strand.

The term “isolated” as used herein refers to a molecule that is substantially separated from its natural environment.

The term “kinetic stabilizer” refers a compound or composition that has the ability to prevent post-secretory dissociation and aggregation of the TTR protein by slowing TTR tetramer dissociation. In certain embodiments, the kinetic stabilizer is a small molecule that occupies a TTR T₄ binding site(s) in order to stabilize the native tetrameric state of TTR over the dissociative transition state, raising the kinetic barrier, imposing kinetic stabilization on the tetramer and preventing amyloidogenesis.

The term “lipolex”, as used herein, refers to a complex comprising a positively-charged cationic lipid (cytofectin) and a nucleic acid. A lipoplex formulation can be used to deliver a nucleic acid agent to cells to induce a desired effect.

The term “local delivery” as used herein refers to refers to delivery of a therapeutic agent directly to a target site within a subject.

The term “microRNA molecule”, “microRNA” or “miRNA”, as used herein, refers to single-stranded RNA molecules, typically of about 21-23 nucleotides in length, which are capable of modulating gene expression. Mature miRNA molecules are partially complementary to one or more messenger RNA (mRNA) molecules, and their main function is to downregulate gene expression.

The term “non-native TTR” as used herein refers to structural TTR conformations that are associated with formation of TTR aggregates including amyloid fibrils.

The term “nucleotide overhang” as used herein refers to at least one unpaired nucleotide that protrudes from the duplex structure of an inhibitory nucleic acid, e.g., a dsRNA. For example, when a 3′-end of one strand of a double-stranded inhibitory nucleic acid extends beyond the 5′-end of the other strand, or vice versa, there is a nucleotide overhang. A double-stranded inhibitory nucleic acid can comprise an overhang of at least one nucleotide; alternatively the overhang can comprise at least two nucleotides, at least three nucleotides, at least four nucleotides, at least five nucleotides or more. A nucleotide overhang can comprise or consist of a nucleotide/nucleoside analog, including a deoxynucleotide/nucleoside. The overhang(s) may be on the sense strand, the antisense strand or any combination thereof Furthermore, the nucleotide(s) of an overhang can be present on the 5′ end, 3′ end or both ends of either an antisense or sense strand of a double-stranded inhibitory nucleic acid. In certain embodiments, the overhang is a 3′ or 5′ overhang on the antisense strand or sense strand.

The term “nucleic acid” or “polynucleotide” as used herein refers to a single or double-stranded polymer of deoxyribonucleotide or ribonucleotide bases read from the 5′ to the 3′ end. The nucleic acid molecule of the present invention encompasses natural nucleic acid, artificial nucleic acid, and mixtures thereof.

The term “on-target event” as used herein means that the TTR mRNA is impacted, i.e., knocked-down, by the RNAi designed to target the TTR gene as evidenced by reduced expression, reduced levels of mRNA, or loss or gain of a particular phenotype. An “on-target” event can be verified via another method such as using a drug that is known to affect the target, or such as rescuing a lost phenotype by introduction of the TTR mRNA from an ortholog, for example.

The term “off-target event” as used herein means that any event other than the desired event in RNA interference.

The term “oligomeric TTR” as used herein, refers to non-native TTR proteins formed by the association of more than 4 TTR monomers. In some embodiments, TTR oligomers are formed of 5-10 subunits, 7-20 subunits, 10-50 subunits, 30-100 subunits, or more than 100 subunits. In some embodiments, TTR oligomers comprise more than 100 subunits. In some embodiments, TTR oligomers comprise less than 8 subunits. In some embodiments, the TTR oligomers are formed of 8, 12, 16, 24, 32, 40, 48, 56, 64, 72, 80, 88, 96, or more subunits. In some embodiments, the TTR oligomers are pentamers, hexamers, heptamers, or octomers. In some embodiments, oligomeric TTR exhibits a molecular weight that is greater than 56 kD. In some embodiments, the subunits in the oligomeric TTR compose of mutant TTR. In some embodiments, the subunits in the said oligomers compose of wild-type TTR. In other embodiments, the subunits in the said oligomers compose of a mixture of mutant and wild-type TTR monomers or truncated monomers.

The term “pharmaceutically acceptable carrier” as used herein refers to a meant, a composition or formulation that allows for the effective distribution of the therapeutic agent to the physical location most suitable for their desired activity. It includes pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

The term “pharmaceutical composition” as used herein is meant to encompass a composition suitable for administration to a subject, such as a mammal, especially a human. In general a “pharmaceutical composition” is preferably sterile, and free of contaminants that are capable of eliciting an undesirable response within the subject (e.g., the compound(s) in the pharmaceutical composition is pharmaceutical grade).

The term “pharmaceutically acceptable salt” of a compound means a salt that is pharmaceutically acceptable and that possesses the desired pharmacological activity of the parent compound.

The term “pharmaceutically acceptable solvate or hydrate” of a compound of the invention means a solvate or hydrate complex that is pharmaceutically acceptable and that possesses the desired pharmacological activity of the parent compound, and includes, but is not limited to, complexes of a compound of the invention with one or more solvent or water molecules, or 1 to about 100, or 1 to about 10, or one to about 2, 3 or 4, solvent or water molecules.

The term “ppercentage (%) sequence identity” with respect to any nucleotide sequence identified herein is defined as the percentage of nucleotide residues in a candidate sequence that are identical with the nucleotide residues in the specific nucleotide sequence, after aligning the sequences and introducing gaps, if necessary, to achieve the maximum percent sequence identity, and not considering any conservative substitutions as part of the sequence identity. Alignment for purposes of determining percentage sequence identity can be achieved in various ways that are within the skill in the art, for instance, using publicly available computer software such as BLAST, BLAST-2, ALIGN or Megalign (DNASTAR) software.

The term “polyplex”, as used herein, refers to a complex comprising a cationic polymer (e.g., polyethylenimines) and a nucleic acid. A lipoplex formulation can be used to deliver a nucleic acid agent to cells to induce a desired effect.

The term “potency” as used herein with reference to an RNAi molecule is a measure of the concentration of an individual or a pool of the RNAi required to knock down TTR mRNA to 50% of the starting mRNA level. Generally, potency is described in terms of IC50, the concentration of the RNAi required for half maximum (50%) mRNA inhibition.

The term “prodrug” as used herein is intended encompass compounds that, under physiological conditions, are converted into the therapeutically active agents of the present invention. A common method for making a prodrug is to include selected moieties that are hydrolyzed under physiological conditions to reveal the desired molecule. In other embodiments, the prodrug is converted by an enzymatic activity of the host animal.

The term “promoter” as used herein refers to a nucleotide sequence that directs the transcription of a structural gene. Typically, a promoter is located in the 5′ non-coding region of a gene, proximal to the transcriptional start site of a structural gene. Sequence elements within promoters that function in the initiation of transcription are often characterized by consensus nucleotide sequences. If a promoter is an inducible promoter, then the rate of transcription increases in response to an inducing agent. In contrast, the rate of transcription is not regulated by an inducing agent if the promoter is a constitutive promoter. Repressible promoters are also known.

The term “rrecombinant” as used herein with reference to an RNA molecule refers to an RNA molecule produced by recombinant DNA techniques; i.e., produced from cells transformed by an exogenous DNA construct encoding the desired RNA.

The term “RNA” as used herein refers to RNA refers to a molecule comprising at least one ribofuranoside moiety. The term can include double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siNA or internally, for example at one or more nucleotides of the RNA. Nucleotides in the RNA molecules of the instant invention can also comprise non-standard nucleotides, such as non-naturally occurring nucleotides or chemically synthesized nucleotides or deoxynucleotides. These altered RNAs can be referred to as analogs or analogs of naturally-occurring RNA.

The term “RNA interference” or “RNAi” refers to a sequence-specific gene silencing mechanism. In the first step, the trigger RNA (either dsRNA or miRNA primary transcript) is processed into a small interfering RNA (siRNA) by the RNase II enzymes Dicer and Drosha. In the second step, siRNAs are loaded into the effector complex RNA-induced silencing complex (RISC). The siRNA is unwound during RISC assembly and the single-stranded RNA hybridizes with mRNA target. Gene silencing is a result of nucleolytic degradation of the targeted mRNA by the RNase H enzyme Argonaute (Slicer). If the siRNA/mRNA duplex contains mismatches the mRNA is not cleaved. Rather, gene silencing is a result of translational inhibition. In addition to siRNA molecules, other interfering RNA molecules and RNA-like molecules can interact with RISC and silence gene expression. Examples of other interfering RNA molecules that can interact with RISC include short hairpin RNAs (shRNAs), single-stranded siRNAs, microRNAs (miRNAs), and dicer-substrate 27-mer duplexes. Examples of RNA-like molecules that can interact with RISC include siRNA, single-stranded siRNA, microRNA, and shRNA molecules containing one or more chemically modified nucleotides, one or more non-nucleotides, one or more deoxyribonucleotides, and/or one or more non-phosphodiester linkages.

All RNA or RNA-like molecules that can interact with RISC and participate in RISC-related changes in gene expression are referred to herein as “interfering RNAs” or “interfering RNA molecules.” SiRNAs, single-stranded siRNAs, shRNAs, miRNAs, and dicer-substrate 27-mer duplexes are, therefore, subsets of “interfering RNAs” or “interfering RNA molecules.”

The term “RNAi molecule” as used herein refers to an RNA molecule that can induce RNA interference in vivo and inhibit the expression of a target gene.

The term “RNA processing” as used herein refers to processing activities performed by components of the siRNA, miRNA or RNase H pathways (e.g., Drosha, Dicer, Argonaute2 or other RISC endoribonucleases, and RNaseH). The term is explicitly distinguished from the post-transcriptional processes of 5′ capping of RNA and degradation of RNA via non-RISC- or non-RNase H-mediated processes.

The term “selectable marker” as used herein refers to any gene which confers a phenotype on a cell in which it is expressed to facilitate the identification and/or selection of cells which are transfected or transformed with a genetic construct. Representative examples of selectable markers include the ampicillin-resistance gene, tetracycline-resistance gene, bacterial kanamycin-resistance gene, zeocin resistance gene, the AURI-C gene which confers resistance to the antibiotic aureobasidin A, phosphinothricin-resistance gene, neomycin phosphotransferase gene (nptII), hygromycin-resistance gene, beta-glucuronidase (GUS) gene, chloramphenicol acetyltransferase (CAT) gene, green fluorescent protein-encoding gene and luciferase gene.

The term “sense strand” as used herein refers to the strand of a dsRNA that includes a region that is substantially complementary to a region of the antisense strand.

The term “short hairpin RNA” or “shRNA” as used herein refers to RNA molecules having an RNA sequence that makes a tight hairpin turn that can be used to silence gene expression via RNA interference. The shRNA hairpin structure is cleaved by the cellular machinery into siRNA, which is then bound to the RNA-induced silencing complex (RISC). This complex binds to and cleaves mRNAs which match the siRNA that is bound to it. shRNA is transcribed by RNA Polymerase III whereas miRNA is transcribed by RNA Polymerase II.

The terms “silence” or “inhibit the expression of,” “down-regulate the expression of,” “suppress the expression of,” and the like, in so far as they refer to the TTR gene, herein refer to the at least partial suppression of the expression of a target gene, as manifested by a reduction of the amount of TTR mRNA which may be isolated from or detected in a first cell or group of cells in which the TTR gene is transcribed and which has or have been treated such that the expression of target gene is inhibited, as compared to a second cell or group of cells substantially identical to the first cell or group of cells but which has or have not been so treated (control cells).

The term “small interference RNA” or “siRNA” as used herein refers to small inhibitory RNA duplexes that induce the RNA interference (RNAi) pathway. The molecule consists of a sense strand (passenger strand) including a nucleotide sequence corresponding to a part of a target gene and an antisense strand (guide strand) thereof siRNAs can be synthetic or processed from double-stranded precursors (dsRNAs) with two distinct strands of base-paired RNA. siRNAs that are derived from repetitive sequences in the genome are called rasiRNAs.

The term “specificity” as used herein with reference to siRNA is a measure of the precision with which a siRNA impacts gene regulation at the mRNA level or protein level, or the true phenotype exhibited by knockdown of the TTR gene function.

The term “subject” has used herein mean a mammal that may have a need for the pharmaceutical methods, compositions and treatments described herein. Subjects and patients thus include, without limitation, primate (including humans), canine, feline, ungulate (e.g., equine, bovine, swine (e.g., pig)), and other subjects. Humans and non-human animals having commercial importance (e.g., livestock and domesticated animals) are of particular interest.

The term “substantially complementary” as used herein refers to sequences of nucleotides where a majority (e.g., at least about 80%, 85%, 90%, 95%, 96%, 97%, 98%, or 99%) or all of the bases in the sequence are complementary, or one or more (e.g., no more than 20%, 15%, 10%, 5%, 4%, 3%, 2%, or 1%) bases are non-complementary, or mismatched.

The term “synergistic” as used herein refers to a combination which is more effective than the additive effects of any two or more single agents. A synergistic effect may permits the effective treatment of a disease using lower amounts (doses) of individual therapy. The lower doses result in lower toxicity without reduced efficacy. In addition, a synergistic effect can result in improved efficacy. Finally, synergy may result in an improved avoidance or reduction of disease as compared to any single therapy.

As used herein, the term “synthetic” refers to a material prepared by chemical synthesis.

The term “systemic delivery” as used herein refers to delivery of a therapeutic agent that leads to broad distribution within the subject. Broad distribution generally requires that the agent is not rapidly degraded or cleared (such as by first pass organs (liver, lung, etc.) or by rapid, nonspecific cell binding) before reaching a disease site distal to the site of administration.

The term “target” is used in a variety of different ways herein and is defined by the context in which it is used, but generally refers to any nucleic acid sequence whose expression or activity is to be modulated “Target RNA” refers to an RNA that would be subject to modulation guided by the antisense strand, such as targeted cleavage or steric blockage. The target RNA could be, for example genomic viral RNA, mRNA, a pre-mRNA, or a non-coding RNA. “Target mRNA” refers to a messenger RNA to which a given RNAi molecule can be directed against. “Target sequence” and “target site” refer to a sequence within the RNA/mRNA to which the antisense strand of an siRNA molecule exhibits varying degrees of complementarity. The phrase “RNAi target” can refer to the gene, mRNA, or protein against which an RNA molecule is directed.

The terms “tetrameric TTR” and “native TTR” are used interchangeably herein to refer to a protein formed by the association of four TTR monomers.

The term “therapeutic agent” or “pharmaceutically active agent” as used herein refers to a compound or other agent that, upon administration to a mammal in a therapeutically effective amount, provides a therapeutic benefit to the mammal.

The term “treating” as used herein means an alleviation, in whole or in part, of symptoms associated with a disorder or disease, or slowing, or halting of further progression or worsening of those symptoms, or prevention or prophylaxis of the disease or disorder in a patient at risk for developing the disease or disorder.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The term “Val30Met” refers to a common TTR gene mutation, involving a substitution of methionine for valine at position 30 in the mature TTR protein (or position 50 in the TTR protein including the signal peptide).

The term “vector” as used herein refers to a nucleic acid molecule capable of mediating entry of (e.g., transferring, transporting, etc.) a second nucleic acid molecule into a cell. The transferred nucleic acid is generally linked to (e.g., inserted into) the vector nucleic acid molecule. A vector may include sequences that direct autonomous replication, or may include sequences sufficient to allow integration into cellular DNA.

### Transthyretin (TTR)

TTR (also known as prealbumin, HsT2651, PALB, and TBPA) transports 15-20% of circulating thyroxine (T4). and the retinol binding protein (RPB). (Blake C. et al., J Mol Biol. 1978; 121:339-356). It circulates in the blood as a 55 kD homo-tetramer. [Klabunde et al., Nat. Struct. Biol. 7:312-321 (2000)]. The concentration in human plasma is between 0.20-0.40 g/L [Hamilton et al., Cell Mol Life Sci 2001, 58:1491-1521].

Each subunit (monomer) is composed of 127 amino acid subunits characterized by two four-stranded anti-parallel β-sheets and a short a-helix. Association of two monomers via their edge beta-strands forms an extended beta sandwich. Further association of two of these dimers in a face-to-face fashion produces the homotetrameric structure and creates the two T4 binding sites per tetramer. The X-ray crystal structure of human TTR is known (Blake, C. et al. (1974) J Mol Biol 88,1-12. 88, 1-12).

The sequence of the mature human TTR protein is:

(SEQ ID NO: 1)
GPTGTGESKC PLMVKVLDAVRGSPAINVAVHVFRKAADDT
WEPFASGKTS ESGELHGLTT EEEFVEGIYK
VEIDTKSYWKALGISPFHEH AEVVFTANDS
GPRRYTIAAL LSPYSYSTTA VVTNPKE.

In one embodiment, the mRNA encoding the mature human TTR protein is predicted to be:

(SEQ ID NO: 2)
ggcccgaccggcaccggcgaaagcaaatgcccgctgatggtgaaagtgct
ggatgcggtgcgcggcagcccggcgattaacgtggcggtgcatgtgtttc
gcaaagcggcggatgatacctgggaaccgtttgcgagcggcaaaaccagc
gaaagcggcgaactgcatggcctgaccaccgaagaagaatttgtggaagg
catttataaagtggaaattgataccaaaagctattggaaagcgctgggca
ttagcccgtttcatgaacatgcggaagtggtgtttaccgcgaacgatagc
ggcccgcgccgctataccattgcggcgctgctgagcccgtatagctatag
caccaccgcggtggtgaccaacccgaaagaa.

A single copy of the 6.8 kbp TTR gene is located on the long arm of chromosome 18 18q11.2-12.1). It has a TATAA box and binding sites for HNF-1, 3 and -4. TTR is expressed primarily in the liver (>95%).

There are numerous TTR-associated diseases, most of which are amyloid diseases. More than 100 destabilizing mutations identified. (Connors L H, et al., 2003. Amyloid. 2003; 10:160-184). Nearly all of these destabilizing mutations are point mutations.

Examples of point mutations associated with AMYL-TTR in the mature TTR protein include 10 (C→R); position 12 (L→P); position 18 (D→E); position 18 (D→G); position 20 (V→I); position 23 (S→N); position 24 (P→S); position 28 (V→M); position 30 (V→L); position 30 (V→M); position 30 (V→G); position 33 (F→I); position 33 (F→L); position 33 (F→V); position 34 (R→T); position 35 (K→N); position 36 (A→P); position 38 (D→V); position 38 (D→A); position 41 (W→L); position 42 (E→D); position 42 (E→G); position 44 (F→S); position 45 (A→T); position 45 (A→S); position 45 (A→D); position 47 (G→E); position 47 (G→A); position 47 (G→R); position 47 (G→V); position 49 (T→I); position 49 (T→A); position 40 (S→R); position 50 (S→I); position 52 (S→P); position 53 (G→E); position 54 (E→K); position 55 (L→Q); position 55 (L→P); position 58 (L→R); position 58 (L→H); position 59 (T→K); position 59 (T→A); position 60 (T→A); position 61 (E→K); position 61 (E→G); position 64 (F→L); position 68 (I→L); position 69 (Y→H); position 70 (K→N); position 71 (V→A); position 73 (I→V); position 77 (S→Y); position 78 (Y→F); position 84 (I→T); position 84 (I→S); position 84 (I→N); position 89 (E→Q); position 89 (E→K); position 91 (A→S); position 97 (A→G); position 97 (A→S); position 106 (T→N); position 107 (I→M); position 107 (I→V); position 111 (L→M); position 114 (Y→C); position 116 (Y→S); position 120 (A→S); position 122 (V→A); position 122 (V→I); position 124 (N→S).

### Composition

In one embodiment, the present invention is a composition comprising an effective amount of least one TTR kinetic stabilizer at least one TTR gene silencer.

(i) TTR Kinetic Stabilizer

Without being bound by any particular theory, it is believed that tetramer dissociation is the initial and rate-limiting step in the TTR amyloidogenesis cascade. (Johnson SM, et al. Acc Chem Res. 2005;38:911-921). In certain embodiments, TTR kinetic stabilizer of the composition of the present invention stabilizes the native tetramer more than the dissociative transition state, thereby raising the kinetic barrier for kinetic barrier for tetramer dissociation, slowing tetramer dissociation, and thus reducing TTR propensity for misfolding and aggregation.

The TTR kinetic stabilizer may be any suitable TTR kinetic stabilizer. In certain embodiments, the TTR kinetic stabilizer is selective and highly potent. In exemplary embodiments, the TTR kinetic stabilizer does not interact with the thyroid hormone receptor (THR) and exhibit minimal NSAID activity.

In one embodiment, the TTR kinetic stabilizer is selected from the group consisting of a small molecule, a peptide, a protein, an antibody, or a polynucleotide. The TTR kinetic stabilizer can be conjugated to a drug, protein, peptide or peptide hormone.

In a particular embodiment, the TTR kinetic stabilizer is a small molecule that stabilizes the native state of transthyretin through tetramer binding, thereby slowing dissociation and amyloidosis under denaturing and physiological conditions through a kinetic stabilization mechanism. In certain embodiments, the small molecule has a molecular weight of less than about 1500 and bind to transthyretin non- or positively cooperatively.

The degree to which the small molecule TTR kinetic stabilizer inhibits TTR aggregation may vary. Inhibition of aggregation can be measured by any suitable assay. (Dolado I. et al. J. Comb. Chem. 7,246-252 (2005); Sekijima Y. et al. Lab. Invest. 83,409-417 (2003)). The TTR may be wild-type TTR (WT-TTR) or mutant TTR (e.g., Y78F-TTR, V1221-TTR, A25T-TTR). In one embodiment, the small molecule inhibits aggregation of TTR by an amount that is about 5%, about 10%, about 15%, about 25%, about 30%, about 35%, about 40%, about 45% or about 50% or greater than tafamidis under the same conditions.

The degree to which the small molecule TTR kinetic stabilizer kinetically stabilizes TTR may vary. Stabilization of TTR can be measured by any suitable assay. (Hurshman A., et al. Biochemistry 43, 7365-7381 (2004)). The TTR may be wild-type TTR (WT-TTR) or mutant TTR (e.g., Y78F-TTR, V1221-TTR). In one embodiment, the small molecule stabilizes TTR by an amount that is about 5%, about 10%, about 15%, about 25%, about 30%, about 35%, about 40%, about 45% or about 50% or greater than tafamidis under the same conditions.

The degree to which the small molecule TTR kinetic stabilizer binds to TTR may vary. Binding to TTR can be measured by any suitable assay. (Almeida M. et al. Biochem. J. 381, 351-356 (2004)). In one embodiment, the small molecule has an affinity for TTR that is about 5%, about 10%, about 15%, about 25%, about 30%, about 35%, about 40%, about 45% or about 50% or greater than tafamidis under the same conditions.

The degree to which the small molecule TTR kinetic stabilizer selectively binds to TTR in plasma may vary. Stabilization of TTR in plasma can be measured by any suitable assay, for example, isoelectric focusing (IF) electrophoresis under semi-denaturing conditions (4 M urea). In one embodiment, the small molecule selectively binds to TTR in plasma with a TTR-binding stoichiometry of about 0.8 or greater, or more particularly, about 0.8, about 0.9, about 1.0, about 1.1, about 1.2, about 1.3, about 1.4, about 1.5 or about 1.6 or greater.

The structure of the small molecule TTR kinetic stabilizer may vary. In one embodiment, TTR kinetic stabilizer is a small molecule comprising two aromatic rings, and more particular, a small molecule comprising two aromatic rings where one bears hydrophilic groups such as an acid or a phenol and the other bears hydrophobic groups such as halogens or alkyls.

In one embodiment, the TTR kinetic stabilizer is selected from the group consisting of COMT inhibitor, a Benzoxazole derivative (e.g., tafamidis), iododiflunisal, diflunisal (or derivatives thereof), resveratrol, tauroursodeoxycholic acid, doxocycline, AG10, or epigallocatechin-3-gallate (EGCG).

In a particular embodiment, the TTR kinetic stabilizer is a COMT inhibitor, i.e., a compound that directly or indirectly inhibits the activity of catechol-O-methyltransferase. COMT inhibitor activity can be determined by methods known in the art, for instance the method disclosed in Zurcher et al (Biomedical Chromatography, 1996, vol. 10, p. 32-36). The COMT inhibitor may be a nucleic acid, a protein (peptide or polypeptide), an analog thereof, a small molecule, or any other agent or chemical that modifies the COMT-encoding nucleic acid, COMT protein, or its activity. The inhibitor may also be a prodrug, meaning it is converted to an COMT inhibitor by metabolic processes.

In one embodiment, the TTR kinetic stabilizer is a small molecule COMT inhibitor selected from the group consisting of tolcapone (Tasmar®), entacapone (Comtan®), opicapone, nitecapone and combinations thereof.

In one embodiment, the TTR kinetic stabilizer is tolcapone or a pharmaceutically acceptable salt thereof.

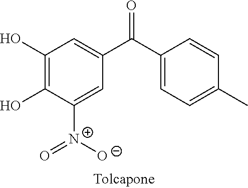 [SMILES: Cc1ccc(C(=O)c2cc(O)c(O)c([N+](=O)[O-])c2)cc1]

Tolcapone is a potent, reversible inhibitor of COMT and the only available COMT inhibitor that is permeable across the blood-brain barrier. It is FDA approved in adult patients for the treatment of Parkinson's disease (PD) as an adjunct therapy with levodopa which is a dopamine precursor and is metabolized by COMT. Tolcapone binds specifically to TTR in human plasma, stabilizes the native tetramer in vivo in mice and humans and inhibits TTR cytotoxicity. (Sant'Anna, R. et al., Nat Commun. 2016; 7: 10787).

(ii) TTR Gene Silencer

The TTR gene silencer may be any suitable gene silencer. In one embodiment, the gene silencer permits post-transcriptional regulation of the TTR gene by RNA interference. In certain embodiments, the TTR gene silencer is an RNAi molecule capable of RNA interference inside a cell or reconstituted in vitro system.

In a particular embodiment., the TTR gene silencer is an RNAi molecule selected from the group consisting of short interfering RNA (siRNA), microRNA (miRNA), and short hairpin RNA (shRNA).

The TTR gene silencer may be, in certain embodiments, provided in the form of a salt, solvate or prodrug.

siRNA Gene Silencer. In one embodiment, the TTR gene silencer is a siRNA molecule. In preferred embodiments, the siRNA has high specificity, high potency, high stability and/or low toxicity.

The siRNA may be a double stranded siRNA (ds siRNA) or single stranded siRNA (ss siRNA). In one embodiment, the siRNA is a double stranded siRNA comprising a guide strand (with antisense complementarity to its mRNA target) base-paired with its passenger (sense) strand. Post-transcriptional gene silencing involves loading of the guide strand into a RISC-loading complex, whereupon the passenger strand is then discarded, and the siRNA guide strand directs RISC to perfectly complementary RNA targets.

The length of the siRNA may vary. In one embodiment, the siRNA is between about 9 and about 35 nucleotides in length. In a particular embodiment, the siRNA is about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34 or about 35 or more nucleotides in length.

In another particular embodiment, the siRNA is between about 15 and about 19, about 15 and 21, about 15 and about 23, about 15 and about 25, about 16 and about 27, about 18 and about 19, about 18 and about 21, about 18 and about 23, about 18 and about 25, about 18 and about 27, about 19 and about 21, about 19 and about 23, about 19 and about 25, about 19 and about 27, about 20 and 21, about 20 and 23, about 20 and 24, about 20 and 25, about 21 and about 23, about 23 and about 25 or about 23 and about 27 nucleotides in length. In a particular embodiment, the siRNA is less than about 30 nucleotides in length.

In one embodiment, the siRNA blunt or blunt ended. In a particular embodiment, the siRNA is a blunt or blunt ended 19-mer, a blunt or blunt ended 21-mer, a blunt or blunt ended 23-mer, a blunt or blunt ended 25-mer or a blunt or blunt ended 27-mer.

In another embodiment, the siRNA has at least one nucleotide overhang. In a particular embodiment, the siRNA has at least one nucleotide overhang at the 3′ end, the 5′ end or combinations thereof. The overhangs may comprise, for example, between about one and about 5 nucleotides, and more particular, one, two, three, four or five nucleotides. In certain embodiments, the overhang is extended (i.e., greater than 5 nucleotides) to permit conjugation of the siRNA molecule to a carrier.

In one embodiment, the siRNA comprises at least one two-nucleotide (2-nt) 3′ overhang.

In a particular embodiment, the siRNA is an asymmetric 19/21 mer, asymmetric 21/23 mer, asymmetric 23/25 mer or asymmetric 25/27-mer.

In another embodiment, the siRNA comprises at least one three-nucleotide (3-nt) 5 overhang.

In a particular embodiment, the siRNA is an asymmetric 19/22 mer, asymmetric 21/24 mer, asymmetric 23/26 mer or asymmetric 25/28-mer.

The identity of the nucleotide overhang may vary. In one embodiment, the overhang comprises one or more ribonucleotides complementary to the target mRNA, including modified or modified ribonucleotides (e.g., 2′-O-methyl modified ribonucleotides).

In another embodiment, the overhang comprises one or more one or more nucleotides that are not ribonucleotides (i.e. non-ribonucleotides). In a particular embodiment, the non-ribonucleotide is a deoxynucleotide, such as deoxy thymidine (dot), deoxyadenosine (day), deoxy guanosine (dog) or deoxy cytosine (DC). In a particular embodiment, the nucleotide overhang includes one, two, three, four or more deoxynucleotides (e.g., did, dada).

When more siRNA contains more than one overhang, the overhangs may be symmetric or asymmetric.

In a particular embodiment, the siRNA contains one or more chemical modifications. In one embodiment, the siNA is a duplex wherein one or both of the strands are chemically modified. In a particular embodiment, the sense strand comprises at least 1, at least 2, at least 3, at least 4, at least 5, at least 6, at least 7, at least 8, at least 9 or at least 10 or more chemical modifications. In another particular embodiment, the antisense strand comprises at least 1, at least 2, at least 3, at least 4, at least 5, at least 6, at least 7, at least 8, at least 9 or at least 10 or more chemical modifications.

The chemical modification may be any suitable modification. In a particular embodiment, the chemical modification is selected from the group consisting of sugar modifications or replacements, base modifications, terminal modifications, backbone modifications or combinations thereof. The modification may improve one or more properties of the siRNA molecule, including without limitation, enhanced nuclease stability, increased binding affinity, or some other beneficial biological property of the siRNA molecule.

The degree of modification may vary. In one embodiment, the siRNA molecule may include from about 5% to about 100% modified nucleotides (e.g., about 5%, 10%, 15%, 20%, 25%, 30%, 35%, 40%, 45%, 50%, 55%, 60%, 65%, 70%, 75%, 80%, 85%, 90%, 95% or 100% modified nucleotides). The actual percentage of modified nucleotides present in a given nucleic acid molecule will depend on the total number of nucleotides present in the nucleic acid. In embodiments where the siRNA is double stranded, the percent modification can be based upon the total number of nucleotides present in the sense strand, antisense strand, or both the sense and antisense strands.

In a particular embodiment, the siRNA comprises one or more ribonucleotides having a sugar modification. The sugar modification may be, for example, a modification on the 2′ moiety of the sugar residue and encompasses amino, fluoro, alkoxy (e.g. methoxy), alkyl, amino, fluoro, chloro, bromo, CN, CF, imidazole, carboxylate, thioate, C1 to C10 lower alkyl, substituted lower alkyl, alkaryl or aralkyl, OCF₃, OCN, O-, S-, or N-alkyl; O-, S-, or N-alkenyl; SOCH₃; SO₂CH₃; ONO₂; NO₂, N₃; heterocycloalkyl; heterocycloalkaryl; aminoalkylamino; polyalkylamino or substituted silyl. In one embodiment, the siRNA comprises one or more ribonucleotides having a 2′O-methyl (methoxy) sugar modification.

In one embodiment, the siRNA comprises at least 1, at least 2, at least 3, at least 4, at least 5, at least 6, at least 7, at least 8, at least 9 or at least 10 or 2′-O-methyl nucleotides.

In one embodiment, the siRNA comprises at least 1, at least 2, at least 3, at least 4, at least 5, at least 6, at least 7, at least 8, at least 9 or at least 10 2′-deoxy-2′-fluoro nucleotides.

In certain embodiments, the siRNA comprises one or more unlocked nucleic acids (UNA). In one embodiment, the RNAi molecule comprises at least one UNA nucleoside, and more particularly, at least 1, at least 2, at least 3, at least 4, at least 5, at least 6, at least 7, or at least 8 UNA nucleosides, for example, from about 2 to about 6 UNA nucleosides, about 3 to about 7 UNA nucleosides, about 4 to about 6 UNA nucleosides or about 3, about 4, about 5, about 6 or about 7 UNA nucleosides.

In a particular embodiment, the siRNA comprises one or more locked nucleic acids (LNA). In LNA, the 2′-hydroxyl oxygen of ribose is connected to the C-4 atom of the same ribose unit via a methylene bridge. In one embodiment, the RNAi molecule comprises at least one LNA nucleoside, and more particularly, at least 1, at least 2, at least 3, at least 4, at least 5, at least 6, at least 7, or at least 8 LNA nucleosides, for example, from about 2 to about 6 LNA nucleosides, about 3 to about 7 LNA nucleosides, about 4 to about 6 LNA nucleosides or about 3, about 4, about 5, about 6 or about 7 LNA nucleosides.

In one embodiment, the siRNA comprises two or more modifications selected from the group comprising unlocked nucleic acid. locked nucleic acid and 2′-O-methylation (OMe). These modifications may be found in the same or different strands (i.e., both antisense and sense).

In a particular embodiment, the siRNA comprises one or more peptide nucleic acids (PNA). PNA is a polymer of purine and pyrimidine bases which are connected to each other via a 2-amino ethyl bridge. In one embodiment, the RNAi molecule comprises at least one PNA nucleoside, and more particularly, at least 1, at least 2, at least 3, at least 4, at least 5, at least 6, at least 7, or at least 8 PNA nucleosides, for example, from about 2 to about 6 PNA nucleosides, about 3 to about 7 PNA nucleosides, about 4 to about 6 PNA nucleosides or about 3, about 4, about 5, about 6 or about 7 PNA nucleosides.

In certain embodiments, the siRNA comprises one or more ribonucleotides having a base modification. In one embodiment, the modification nucleobase is selected from the group consisting of 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine.

In certain embodiments, the siRNA comprises one or more backbone modifications. In a particular embodiment, the backbone modification is a phosphate backbone modification selected from the group consisting of phosphorothioate, phosphonoacetate, and/or thiophosphonoacetate, phosphorodithioate, methylphosphonate, phosphotriester, morpholino, amidate carbamate, carboxymethyl, acetamidate, polyamide, sulfonate, sulfonamide, sulfamate, formacetal, thioformacetal, and/or alkylsilyl, substitutions or a combination thereof.

In a particular embodiment, the siRNA contains one or more terminal modifications. Such terminal modifications may include, for example, addition of a nucleotide, a modified nucleotide, a lipid, a peptide, a sugar and inverted abasic moiety. Such modifications are incorporated, for example at the 3′ and/or 5′ terminus of the siRNA.

The target sequence may be a coding sequence, non-coding sequence, or both coding and non-coding sequences. In a particular embodiment, the target sequence comprises at least one region of an mRNA encoding a TTR protein having one or more amino acid substitutions, insertions or deletions. In one embodiment, the target sequence is an mRNA encoding a TTR protein with a Val30Met mutation. In another embodiment, the target sequence is an mRNA encoding a TTR protein with T60A (Ala 60) mutation. In a further embodiment, the target sequence is an mRNA encoding a TTR protein with a Val122Ile mutation. In yet another embodiment, the target sequence is a TTR protein with a Val50Met mutation.

In one embodiment, the siRNA is fully complementary to the target sequence. In another embodiment, the siRNA is substantially complimentary to the target sequence.

In a particular embodiment, the siRNA is about 80%, about 85%, about 90%, about 91%, about 92%, about 93%, about 94%, about 95%, about 96%, about 97%, about 98%, about 99% or about 100% complimentary to the target sequence.

In another particular embodiment, the siRNA is about 80%, about 85%, about 90%, about 91%, about 92%, about 93%, about 94%, about 95%, about 96%, about 97%, about 98%, about 99% or about 100% complimentary to the target sequence over a region about 14 to about 32 nucleotides in length. In one embodiment, the siRNA is about 80%, about 85%, about 90%, about 91%, about 92%, about 93%, about 94%, about 95%, about 96%, about 97%, about 98%, about 99% or about 100% complimentary to the target sequence over a region about 16 to about 28 nucleotides in length, about 18 and about 26 nucleotides in length, or about 18, about 19, about 20, about 21, about 22, about 23, about 24, about 25 or about 26 nucleotides in length. In a particular embodiment, the siRNA is about 80%, about 85%, about 90%, about 91%, about 92%, about 93%, about 94%, about 95%, about 96%, about 97%, about 98%, about 99% or about 100% complimentary to the target sequence over a region greater than about 24 nucleotides in length, greater than about 26 nucleotides in length or greater than about 28 nucleotides in length.

In one embodiment, the siRNA contains no more than 3 mismatches with the target sequence. In another embodiment, the siRNA contains no more than 5 mismatches with the target sequence.

In one embodiment, the siRNA contains more than 1 mismatch with the target sequence but preferentially in the central region of the siRNA.

In one embodiment, the siRNA is fully complimentary to an mRNA encoding SEQ ID NO:1 or a fragment thereof.

In another embodiment, the siRNA is substantially complementary to an mRNA sequence encoding SEQ ID NO: 1 or a fragment thereof.

In a particular embodiment, the target sequence is an mRNA encoding a fragment of SEQ ID NO: 1, wherein the fragment is between about 14 to about 32 nucleotides, about 14 and about 30 nucleotides, about 14 and about 28 nucleotides, about 14 and about 26 nucleotides, about 14 and about 24 nucleotides, about 14 and about 22 nucleotides, about 14 and about 20 nucleotides, about 14 and about 18 nucleotides, about 14 and about 16 nucleotides; about 16 and about 32 nucleotides, about 16 and about 30 nucleotides, about 16 and about 28 nucleotides; about 16 and about 26 nucleotides, about 16 and about 22 nucleotides, about 16 and about 20 nucleotides, about 16 and about 18 nucleotides; about 20 and about 32 nucleotides; about 20 and about 30 nucleotides; about 20 and about 28 nucleotides; about 20 and about 26 nucleotides; about 20 and about 24 nucleotides; about 22 and about 24 nucleotides; about 22 and about 32 nucleotides; about 22 and about 30 nucleotides; about 24 and about 28 nucleotides; about 24 and about 26 nucleotides; about 26 and about 32 nucleotides; about 26 and about 30 nucleotides; about 26 and about 38 nucleotides; about 28 and about 32 nucleotides; about 28 and about 30 nucleotides. In another particular embodiment, the target sequence is an mRNA encoding a fragment of SEQ ID NO: 1, wherein the fragment is about 30 nucleotides; about 28 nucleotides; about 26 nucleotides; about 24 nucleotides; about 22 nucleotides; about 20 nucleotides; about 18 nucleotides; about 16 nucleotides or about 14 nucleotides.

In one embodiment, the siRNA is U-rich and depleted in G. In a particular embodiment, the siRNA is an isolated, double-stranded siRNA having one or more of the following characteristics: (i) A/U at the 5′ end; (ii) AU-richness in the 5′ terminal 7 bp region of the antisense strand; (ii) G/C at the 5′ end of the sense strand; and (ii) the absence of any long GC stretch of more than 9 bp in length.

The siRNAcan be employed in a variety of pharmaceutically acceptable salts. Such salts may include non-toxic organic or inorganic acids. For example, those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In one embodiment, the siRNA is an isolated double-stranded siRNA, wherein (a) each strand of the siRNA molecule is about 9 to about 35 nucleotides in length, or more particularly, about 14 to about 32, about 18 to about 27 nucleotides, about 18 to about 24 nucleotides or about 19 to about 23 nucleotides in length; and (b) one strand of the siRNA molecule comprises a ribonucleotide sequence substantially complimentary to an mRNA encoding SEQ ID. NO: 1 or a fragment thereof. In a particular embodiment, the fragment is less than about 30 nucleotides, less than about 28 nucleotides, less than about 26 nucleotides, less than about 24 nucleotides or less than about 22 nucleotides but in each case, greater than three nucleotides.

In a particular embodiment, the siRNA is an isolated double-stranded siRNA, wherein (a) each strand of the siRNA molecule is about 9 to about 35 nucleotides in length, or more particularly, about 14 to about 32, about 18 to about 27 nucleotides, about 18 to about 24 nucleotides or about 19 to about 23 nucleotides in length; and (b) one strand of the siRNA molecule comprises a ribonucleotide sequence fully complimentary an mRNA encoding SEQ ID. NO: 1 or a fragment thereof. In a particular embodiment, the fragment is less than about 30 nucleotides, less than about 28 nucleotides, less than about 26 nucleotides, less than about 24 nucleotides or less than about 22 nucleotides but in each case, greater than three nucleotides.

In one embodiment, the siRNA is an isolated double-stranded siRNA, wherein (a) each strand of the siRNA molecule is about 9 to about 35 nucleotides in length, or more particularly, about 14 to about 32, about 18 to about 27 or about 19 to about 23 nucleotides in length; and (b) one strand of the siRNA molecule comprises a ribonucleotide sequence at least about 80, at least about 85, at least about 90, at least about 95, at least about 96, at least about 97, at least about 98, at least about 99% complimentary to an mRNA encoding SEQ ID. NO: 1 or a fragment thereof.

In one embodiment, the siRNA is chemically synthesized. The siRNA may be chemically synthesized by methods known in the art, for example, using an automated synthesizer or generated by cleavage of a longer dsRNA. The chemically synthesized siRNA may be administered in its native form, optionally in combination with a pharmaceutically acceptable carrier. In certain embodiments, a longer version of the siRNA is administered (siRNA+), which is then further processed in vivo to provide the mature siRNA.

In another embodiment, the siRNA is produced by an expression vector. In a particular embodiment, the siRNA is provided as a vector that expresses an shRNA, which is further processed into an siRNA in the cytoplasm. The vector may be any suitable vector such as a plasmid or viral vector (e.g., a retroviral, including lentiviral, adenoviral, baculoviral, and avian viral vector).

In one embodiment, the invention provides expression vector comprising a promoter and a sequence encoding an shRNA. The transcription promoter is operably linked, either directly or indirectly to the gene and selected based on the host cell and the effect sough. It may be, for example, a constitutive or inducible promoter. In a particular embodiment, the promoter is an RNA polymerase III promoter (e.g., H1 or U6).

In a particular embodiment, the siRNA is highly stable under a variety of conditions pertinent to storage, delivery, and/or use. Stability can be measured by any suitable means, including (for example), nondenaturing PAGE.

In a particular embodiment, the siRNA is highly activity under a variety of conditions pertinent to storage, delivery and/or use. Activity can be measured by any suitable method, including for example, a bioluminescence (luciferase)-based tissue culture assay.

In one embodiment, the siRNA is highly stable and/or retains high activity when stored at C or 21° C. for 4 weeks or alternatively at 37° C. for 5 and 24 hours or 95° C. for 10, 30, 60, and 120 minutes.

In another embodiment, the siRNA is highly stable and/or retains high activity when incubated in various biological fluids (e.g., human serum) at 37° C. for 10 minutes, 1 hour, 5 hours, and 48 hours.

The siRNA gene silencer may be, in certain embodiments, provided in the form of a salt, solvate or prodrug. miRNA gene silencer. In one embodiment, TTR gene silencer is an miRNA molecule. In preferred embodiments, the miRNA has high specificity, high potency, high stability and/or low toxicity.

In a particular embodiment, the miRNA does not significantly elicit off-target effects and/or an interferon response. In another particular embodiment, the miRNA does not significantly interference in endogenous miRNA biogenesis.

The length of the miRNA molecule may vary. In one embodiment, the miRNA molecule is between about 9 and about 35 nucleotides. In a particular embodiment, the siRNA is about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34 or about 35 or more nucleotides. In another particular embodiment, the miRNA is between about 15 and about 19 nucleotides, about 15 and 21 nucleotides, about 15 and about 23 nucleotides, about 15 and about 25 nucleotides, about 16 and about 27 nucleotides, about 18 and about 19 nucleotides, about 18 and about 21 nucleotides, about 18 and about 23 nucleotides, about 18 and about 25 nucleotides, about 18 and about 27 nucleotides, about 19 and about 21 nucleotides, about 19 and about 23 nucleotides, about 19 and about 25 nucleotides, about 19 and about 27 nucleotides, about 21 and about 23 nucleotides, about 21 and about 25 nucleotides, about 21 to about 27 nucleotides, about 23 to about 25, or about 23 to about 27 nucleotides. In a particular embodiment, the miRNA is less than about 30 nucleotides.

In one embodiment, the miRNA has blunt ends, i.e., the miRNA has no 3′ or 5′ overhang.

In another embodiment, the miRNA has one or more nucleotides overhanging on the 5′ end or the 3′ end of either strand of the miRNA. The overhangs may comprise, for example, between about one and about 5 nucleotides, and more particular, one, two, three, four or five nucleotides. In a particular embodiment, the overhang nucleotides are dinucleotides.

In a particular embodiment, the miRNA comprises a 2 nt, 3′ overhang.

In one embodiment, the miRNA molecule has one or more chemical modifications. The chemical modification may be any suitable modification. In a particular embodiment, the chemical modification is selected from the group consisting of sugar modifications or replacements, base modifications, terminal modifications, backbone modifications or combinations thereof. The modification may improve one or more properties of the miRNA molecule, including without limitation, enhanced nuclease stability, increased binding affinity, or some other beneficial biological property of the miRNA molecule.

The degree of modification may vary. In one embodiment, the miRNA molecule may include from about 5% to about 100% modified nucleotides (e.g., about 5%, 10%, 15%, 20%, 25%, 30%, 35%, 40%, 45%, 50%, 55%, 60%, 65%, 70%, 75%, 80%, 85%, 90%, 95% or 100% modified nucleotides).

The chemical modification may be, for example, any chemical modification taught herein for use with siRNA molecules—above.

In a particular embodiment, the miRNA comprises phosphorothioates and 2′-O-methyl modified sequences.

The target sequence may be a coding sequence, non-coding sequence, or both coding and non-coding sequences. In one embodiment, the target sequence is at least one region of an mRNA encoding a normal TTR protein. In another embodiment, the target sequence is at least one region of an mRNA encoding a mutant TTR protein. In a particular embodiment, the target sequence is at least one region of an mRNA encoding a TTR protein one or more amino acid substitutions, insertions or deletions—including any mutant TTR protein described herein.

In one embodiment, the target sequence is an mRNA encoding a TTR protein with a Val30Met mutation. In another embodiment, the target sequence is an mRNA encoding a TTR protein with T60A (Ala 60) mutation. In a further embodiment, the target sequence is an mRNA encoding a TTR protein with a Val122Ile mutation

Without being bound by any particular theory, it is believed that the extent of sequence complementarity between the miRNA guide strand and the mRNA target determines whether translation arrest or mRNA cleavage results from mRNA recognition by RISC. Generally, the higher the degree of complementarity, the more likely cleavage is the mechanism.

In one embodiment, the miRNA is substantially complementary to the target mRNA. In another embodiment, the miRNA is fully complimentary to the target mRNA.

In a particular embodiment, the miRNA is about 80%, about 85%, about 90%, about 91% about 92%, about 93%, about 94%, about 95%, about 96%, about 97%, about 98%, about 99% or about 100% complementary to the target sequence.

In a particular embodiment, the miRNA is about 80%, about 85%, about 90%, about 91% about 92%, about 93%, about 94%, about 95%, about 96%, about 97%, about 98%, about 99% or about 100% complementary to the target sequence across a region of 14 about 32 nucleotides.

In a particular embodiment, the miRNA is about 80%, about 85%, about 90%, about 91% about 92%, about 93%, about 94%, about 95%, about 96%, about 97%, about 98%, about 99% or about 100% complementary to the target sequence across a region of about 16 to about 30 nucleotides, about 18 to about 28 nucleotides, about 20 to about 26 nucleotides, or about 18, about 19, about 20, about 21, about 22, about 23, about 24, about 25, about 26, about 27 or about 28 or more nucleotides.

In particular embodiment, the miRNA is about 80%, about 85%, about 90%, about 91% about 92%, about 93%, about 94%, about 95%, about 96%, about 97%, about 98%, about 99% or about 100% complementary to the target sequence across a region of less than about 30, less than about 28, less than about 26, less than about 24 nucleotides in length but in each case, greater than three nucleotides.

In one embodiment, the miRNA is administered in native form and optionally, associated with a pharmaceutical carrier.

In another embodiment, the miRNA is administration in the form of a vector comprising a sequence encoding the miRNA which is then transcribed in vivo. The vector may be any suitable vector including, for example, a plasmid or viral vector (e.g., an adenovirus, an adeno-associated virus, a retrovirus or lentivirus). Thus, in one embodiment, the invention provides expression vector comprising a sequence encoding one or more miRNAs capable of modulating the expression of the TTR gene. The expression vector may be any suitable vector, for example, a plasmid vector or a viral vector (e.g., a lentiviral vector). In one embodiment, the expression vector comprises a transcription promoter, a gene encoding the miRNA and a transcription terminator. The transcription promoter is operably linked, either directly or indirectly to the gene and selected based on the host cell and the effect sought. It may be, for example, a constitutive or inducible promoter. In a particular embodiment, the promoter is RNA II polymerase promoter.

In a particular embodiment, the promoter is an inducible promoter selected from the group consisting of tetracycline-inducible promoters, PIT-inducible promoters, tetracycline trans activator systems, and reverse tetracycline trans activator (rattan) systems.

In one embodiment, the miRNA is provided as a prim-miRNA or a variant thereof. The prim-miRNA sequence may comprise from 45-250, 55-200, 70-150 or 80-100 nucleotides. The pri-mRNA may be cleaved DROSHA/DGCR8 to provide a hairpined structure known as a pre-miRNA. The pre-miRNA is further processed to produce a miRNA duplex (miRNA/miRNA) with the hairpin removed. The duplex is then incorporated into an RISC, wherein the stable associated guide strand remains in the RISC, while the passenger strand is generally released and cleaved. The e RISC complex subsequently finds cellular mRNAs partially complementary to the loaded guide strand sequence and prevents translation, either via translational arrest or mRNA cleavage. A single pri-miRNA may contain from one to several miRNA precursors.

In one embodiment, the miRNA is expressed in an amount sufficient to attenuate TTR gene expression in a sequence specific manner. In a preferred embodiment, the miRNA is stably expressed in the mammalian cell.

The miRNAcan be employed in a variety of pharmaceutically acceptable salts. Such salts may include non-toxic organic or inorganic acids. For example, those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

The miRNA gene silencer may be, in certain embodiments, provided in the form of a salt, solvate or prodrug.

shRNA gene silencer. In one embodiment, the TTR gene silencer is a shRNA. In preferred embodiments, the siRNA has high specificity, high potency, high stability and/or low toxicity.

In a particular embodiment, the shRNA comprises an antisense (lower) and a sense strands (upper) connected by a loop of unpaired nucleotides. The shRNA stem is defined as the stretch of sequence between the terminally paired nucleotides. The stem may comprise a perfectly complementary or may container one or more bulges or interior loops. In one embodiment, the antisense strand contains at least one mismatch, and in particular, at least one substitution, deletion or insertion, as long as the antisense strand and the sense strand can hybridize under stringent conditions.

In a particular embodiment, the shRNA does not significantly elicit off-target effects and/or an interferon response. In another particular embodiment, the shRNA does not significantly interference in endogenous shRNA biogenesis.

The shRNA may vary in length. In one embodiment, the shRNA is between about 40 and 100 nucleotides in length, or more particularly, about 40 and about 70 nucleotides, and even more particularly, about 40 and about 60 nucleotides, about 35 and about 55 or about 40 and about 50 nucleotides in length. In a particular embodiment, the shRNA is about 40, about 45, about 50, about 55, about 60, about 65, about 70, about 75, about 80, about 85, about 90 or about 100 nucleotides in length.

The length of the shRNA stem may vary. In a particular embodiment, the shRNA stem is between about 15 and about 50 nucleotides in length, or more particularly, about 20 and about 45, about 25 and about 35 or about 30 nucleotides in length. In a particular embodiment, the shRNA is about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39 or about 40 nucleotides in length.

In one embodiment, the shRNA stem is between about 15 and about 20, about 16 and about 22, about 18 and about 24, about 20 and about 26, about 22 and about 28, about 24 and about 30, about 26 and about 32 or about 28 and about 34 nucleotides in length.

In one embodiment, the shRNA stem is less than about 20 nucleotides.

In a particular embodiment, the shRNA has a loop of at least about 4, at least about 5, at least about 6, at least about 7 or at least about 8 or more nucleotides.

In one embodiment, the shRNA is blunt ended, i.e., has no 3′ or 5′ overhang.

In another embodiment, the shRNA has one or more nucleotides overhanging on the 5′ end and/or the 3′ end of either strand of the miRNA. The overhangs may comprise, for example, between about one and about 5 nucleotides, and more particular, one, two, three, four or five nucleotides. In a particular embodiment, the overhang nucleotides are dinucleotides.

In one embodiment, the shRNA comprises 5′ overhang, a targeting sequence, loop, reverse-complement targeting sequence, transcriptional terminator sequence, and 3′ overhang. In a particular embodiment, the overhang at the 5′ end is a 2-nucleotide (nt) or 3-nt overhang. In another particular embodiment, the overhang at the 3′ end is a 2-nt or 3-nt overhang.

The shRNA may have one or more chemical modifications, in either or both strands. The chemical modification may be any suitable modification. In a particular embodiment, the chemical modification is selected from the group consisting of sugar modifications or replacements, base modifications, terminal modifications, backbone modifications or combinations thereof. The modification may improve one or more properties of the shRNA molecule, including without limitation, enhanced nuclease stability, increased binding affinity, or some other beneficial biological property of the shRNA molecule.

The degree of modification may vary. In one embodiment, the shRNA molecule may include from about 5% to about 100% modified nucleotides (e.g., about 5%, 10%, 15%, 20%, 25%, 30%, 35%, 40%, 45%, 50%, 55%, 60%, 65%, 70%, 75%, 80%, 85%, 90%, 95% or 100% modified nucleotides).

The modification may be any suitable modification including, but not limited to, those described above with respect to siRNA.

The shRNAcan be employed in a variety of pharmaceutically acceptable salts. Such salts may include non-toxic organic or inorganic acids. For example, those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

The target sequence may be a coding sequence, non-coding sequence, or both coding and non-coding sequences. In one embodiment, the target sequence is at least a portion of an mRNA encoding as normal (i.e., native) TTR protein. In another embodiment, the target sequence is at least a portion of an mRNA encoding a mutant (i.e., non-native) TTR protein. In a particular embodiment, the target sequence corresponds to a TTR protein having one or more amino acid substitutions, insertions or deletions—including any mutant TTR protein described herein

In one embodiment, the target sequence is an mRNA encoding a TTR protein with a Val30Met mutation. In another embodiment, the target sequence is an mRNA encoding a TTR protein with T60A (Ala 60) mutation. In a further embodiment, the target sequence is an mRNA encoding a TTR protein with a Val122Ile mutation

The shRNA can be administered in native form, optionally in combination with a pharmaceutical carrier.

Alternatively, the sHRNA can be provided in the form of a vector comprising a sequence encoding the shRNA which is then transcribed in vivo. The vector may be any suitable vector including, for example, a plasmid or viral vector (e.g., an adenovirus, an adeno-associated virus, a retrovirus or lentivirus). Thus, in one embodiment, the invention provides expression vector comprising a sequence encoding one or more shRNAs capable of modulating the expression of the TTR gene. The expression vector may be any suitable vector, for example, a plasmid vector or a viral vector (e.g., a lentiviral vector). In one embodiment, the expression vector comprises a transcription promoter, a gene encoding the sHRNA and a transcription terminator. The transcription promoter is operably linked, either directly or indirectly to the gene and selected based on the host cell and the effect sought. It may be, for example, a constitutive or inducible promoter. In a particular embodiment, the promoter is selected from U6, H1, CMV, PGK, and UbiC.

In one embodiment, the shRNA is provided in the in the form of artificial pri-miRNA transcripts, i.e., embedded into a miRNA context such as the miR-30 stem loop precursor.

In another embodiment, invention provides an expression vector comprising a sequence encoding one or more shRNAs capable of modulating the expression of the TTR gene. In one embodiment, the expression vector comprises a transcription promoter, a gene encoding the shRNA and a transcription terminator. The transcription promoter is operably linked, either directly or indirectly to the gene and selected based on the host cell and the effect sough. It may be, for example, a constitutive or inducible promoter. In a particular embodiment, the promoter is a RNA polymerase III promoter.

The shRNA gene silencer may be, in certain embodiments, provided in the form of a salt, solvate or prodrug.

(iii) Other Pharmaceutically Active Agents

In certain embodiments, the composition may further comprise or be co-administered with one or more pharmaceutically active agents, including an anti-amyloid or anti-fibril agent.

In one embodiment, the composition may further comprise or be co-administered with a protein stabilizing agent, such as resveratrol, heat shock proteins, protein chaperones, and mimics thereof.

In other embodiments, the composition may further comprise or be co-administered in combination with other therapies for diseases caused by TTR amyloid fibrils. Therapies for diseases caused by TTR amyloid fibrils include heart transplant for TTR cardiomyopathy, liver transplant, Tafamidis treatment, and the like.

The compound described above may be administered before, after, or during another therapy for diseases caused by TTR amyloid fibrils.

(iv) Other Components

The TTR kinetic stabilizer and/or TTR gene silencer used in the method of the present invention can be administered alone or together with a pharmaceutical carrier selected with respect to the intended form of administration and as consistent with conventional pharmaceutical practices. Suitable pharmaceutical carriers are described in Remington's Pharmaceutical Sciences, Mack Publishing Co., Easton, Pa., latest edition. The pharmaceutical compositions may be specifically formulated for administration by any suitable route such as oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route. The therapeutic agents can be administered in liquid or solid form.

In one embodiment, one or both of the therapeutic agents (i.e., the TTR kinetic stabilizer and/or TTR gene silencer) may be administered intravenously or intraperitoneally by infusion or injection. Solutions of the therapeutic agent or its salts may be prepared in water or saline, optionally mixed with a non-toxic surfactant. Dispersions may be prepared in glycerol, liquid polyethylene glycols, triacetin, and mixtures thereof, and in oils. Under ordinary conditions of storage and use, these preparations contain a preservative to prevent growth of microorganisms.

Pharmaceutical dosage forms suitable for injection or infusion may include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form is optionally sterile, fluid, and stable under conditions of manufacture and storage. The liquid carrier or vehicle may be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof.

In one embodiment, one or both of the therapeutic agents (i.e., the TTR kinetic stabilizer and/or TTR gene silencer) may be combined with one or more excipients and used in the form of ingestible tablets, buccal tablets, troches, capsules, elixirs, suspensions, syrups, wafers, and the like. Such compositions and preparations can contain at least 0.1% (w/w) of at least one therapeutic agents. The percentage of the compositions and preparations can, of course, be varied, for example from about 0.1% to nearly 100% of the weight of a given unit dosage form. The amount of the at least one therapeutic agent is such that an effective dosage level will be obtained upon administration.

The tablets, troches, pills, capsules, and the like may also contain one or more of the following: binders, such as microcrystalline cellulose, gum tragacanth, acacia, corn starch, or gelatin; excipients, such as dicalcium phosphate, starch or lactose; a disintegrating agent, such as corn starch, potato starch, alginic acid, primogel, and the like; a lubricant, such as magnesium stearate or Sterotes; a glidant, such as colloidal silicon dioxide; a sweetening agent, such as sucrose, fructose, lactose, saccharin, or aspartame; a flavoring agent such as peppermint, methylsalicylate, oil of wintergreen, or cherry flavoring; and a peptide antibacterial agent, such as envuvirtide (Fuzeon™). When the unit dosage form is a capsule, it can contain, in addition to materials of the above type, a liquid carrier, such as a vegetable oil or a polyethylene glycol. Various other materials may be present as coatings or to otherwise modify the physical form of the solid unit dosage form.

In one embodiment, one or both of the therapeutic agents are prepared with carriers that will protect the compound against rapid elimination from the body, such as a controlled release formulation, including implants and microencapsulated delivery systems. Biodegradable, biocompatible polymers may be used, such as modified conjugated cellulose, ethylene vinyl acetate, polyanhydrides, polyglycolic acid, collagen, polyorthoesters, and polyacetic acid.

In one embodiment, the TTR kinetic stabilizer and the TTR gene silencer are co-administered systemically. In a particular embodiment, the TTR kinetic stabilizer is administered orally, while the TTR gene silencer is administered by intravenous or intraperitoneal delivery. In another embodiment, the TTR kinetic stabilizer is administered systemically, e.g., orally, while the TTR gene silencer is administered locally.

In one embodiment, the RNAi molecule is administered naked, i.e., not conjugated to, encapsulated by or complexed with a carrier. This embodiment generally requires one or more chemical modifications to protect the RNAi molecule from degradation. Any suitable chemical modification can be used, including, but not limited to, those chemical modifications disclosed herein.

The relationship between the RNAi molecule and the carrier may vary, including covalent and non-covalent association. In a particular embodiment, the RNAi molecule is encapsulated in a nanoparticulate formulation. In another particular embodiment, the RNAi molecule is complexed with the carrier, e.g., on the basis of charge. In yet another particular embodiment, the RNAi is conjugated to the carrier.

In one embodiment, the RNAi molecule is conjugated to a molecule intended to do one or more of the following: enhance cell targeting, prolong drug circulation time and/or improve cell membrane permeation. In one embodiment, the RNAi molecule is conjugated to a small molecule, lipid, peptide, protein, polymer or nucleic acid.

In a particular embodiment, the RNAi molecule is conjugated to a lipid. In one embodiment, the lipid is selected from the group consisting of cholesterol, bile acids, long chain fatty acids or α-tocopherol.

In another particular embodiment, the RNAi molecule is conjugated to a peptide. In one embodiment, the peptide is a cell-penetrating peptide (CPP). CPPs are small peptides, typically between about 7 and about 30 amino acids, and generally comprise high content of basic amino acids. In a particular embodiment, the CPP is selected from the group consisting of Tat, transportan, penetratin, polyarginine peptide Arg₈ sequence, VP22 protein from Herpes Simplex Virus (HSV), antimicrobial peptides Buforin I and SynB and polyproline sweet arrow peptide.

In a further embodiment, the RNAi molecule is conjugated to a heavy-chain antibody fragment (FAB), e.g., via a protamine linker.

In another embodiment, the RNAi molecule is conjugated to polymer.

In one embodiment, the RNAi molecule is conjugated to PEG. In a particular embodiment, the RNAi is formulated as a polyelectrolyte complex (PEC) micelle.

In another embodiment, the RNAi molecule is conjugated to an aptamer, e.g., an RNA aptamer.

In other embodiments, the RNAi molecule is not covalently conjugated to the carrier. The carrier may be, for example, a nanocarrier.

In a particular embodiment, the RNAi molecule is encapsulated by the carrier. In certain embodiments, the RNAi may be encapsulated by a liposome. Liposomes are vesicular structures that can form via the accumulation of lipids interacting with one another in an energetically favorable manner. The lipid component of the liposome may be cationic lipids, fusogenic lipids, polyethylene glycosylated (PEG) lipids, cholesterol or a combination thereof In a particular embodiment, the liposome is coated with PEG.

In another embodiment, the RNAi molecule is complexed with a cationic lipid, e.g., on the basis of charge, where the RNAi molecule is anionic and the carrier is cationic or comprises a cationic molecule. In a particular embodiment the cationic carrier is a cationic lipid, a cationic peptide or a cationic polymer.

In a particular embodiment, the RNAi molecule is complexed with a mixture of cationic and fusogenic lipids, coated with diffusible polyethylene glycol, to provide a stable nucleic acid-lipid particle (SNALP). In one embodiment, the SNALP is delivered systemically. In another particular embodiment, the RNAi molecule is complexed with cholesterol and PEG-modified lipids to provide a lipidoid nanoparticles.

In one embodiment, the RNAi molecule is complexed with a cationic peptide, including, but not limited to, atelocollagen, poly(l-lysine) or protamine.

In another particular embodiment, the RNAi molecule is complexed with a cationic polymer including, but not limited to, polyethylenimine (PEI). In a particular embodiment, the RNAi is complexed with cyclodextrin to provide a cyclodextrin polymer nanoparticle. In another particular embodiment, the RNAi is complexed with a phospholipid and low molecular weight PEI to provide a micelle-like nanoparticle (MNP).

In one embodiment, the RNAi is formulated for targeted delivery, i.e., to a tissue, cell or subcellular location of interest. In a particular embodiment, the RNAi is formulated for delivery to the liver. In a particular embodiment, the RNAi s is formulated as a lipid nanoparticle comprising polyethylene glycol-conjugated (PEGylated) lipids, cholesterol and nucleic acids. The lipid nanoparticular may be, for example, between about 50-100 nm in diameter. In another particular embodiment, the RNAi is formulated as a GalNAc conjugate, i.e., attached to N-acetylgalactosamine (GalNAc).

In another embodiment, the carrier is a viral vector. Representative, non-limiting viral vectors include retroviral, adenovirus, adenovirus-associated, slow virus, and herpes simplex virus vectors.

In certain embodiments, the TTR kinetic stabilizer and the TTR gene silencer are formulated for single dosage administration. In other embodiments, the TTR kinetic stabilizer and the TTR gene silencer are formulated for multiple dosage administration.

The amount of active ingredient that may be combined with the carrier materials to produce a single dosage form will vary depending upon the host treated, and the particular mode of administration. It should be understood, however, that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of active ingredient may also depend upon the therapeutic or prophylactic agent, if any, with which the ingredient is co-administered.

### Methods

In one embodiment, the invention provides a method of manufacturing the RNAi molecule disclosed herein. In a particular embodiments, the method comprising reacting nucleotide units and thereby forming covalently linked contiguous nucleotide units comprising the RNAi.

In on embodiment, the invention provides a method of preventing transthyretin-associated amyloidosis comprising administering a prophylactically effective amount of the composition disclosed herein to a subject in need thereof, e.g., a human.

In one embodiment, the invention provides a method of treating transthyretin-associated amyloidosis comprising administering a therapeutically effective amount of the composition disclosed herein to a subject in need thereof, e.g., a human.

As one of skill in the art would understand, TTR-associated amyloidosis is a slowly progressive condition characterized by the buildup of abnormal deposits amyloid (amyloidosis) in the body's organs and tissues. The process of amyloidosis is linked to tissue degeneration, yet amyloid fibrils themselves may not mediate the cytotoxicity. Local cellular activation, ultimately resulting in cell dysfunction and death, may contribute to the pathogenesis.

There are three major forms of transthyretin amyloidosis: neuropathic, cardiac and leptomeningeal. These forms can affect a wide range tissues and organs.

The neuropathic form of transthyretin amyloidosis primarily affects the peripheral and autonomic nervous systems, resulting in peripheral neuropathy and difficulty controlling bodily functions. Impairments in bodily functions can include sexual impotence, diarrhea, constipation, problems with urination, and a sharp drop in blood pressure upon standing (orthostatic hypotension). Some people experience heart and kidney problems as well. Various eye problems may occur, such as cloudiness of the clear gel that fills the eyeball (vitreous opacity), dry eyes, increased pressure in the eyes (glaucoma), or pupils with an irregular or “scalloped” appearance. Some people with this form of transthyretin amyloidosis develop carpal tunnel syndrome, which is characterized by numbness, tingling, and weakness in the hands and fingers.

In one embodiment, the neuropathic TTR-associated amyloidosis is familial amyloid polyneuropathy (FAP) (also called hereditary ATTR-polyneuropathy, abbreviated also as hATTR-polyneuropathy, or Corino de Andrade's disease). It is an autosomal dominant neurodegenerative disease, which manifests as amyloid deposition accumulates over time and typically, between about 20 and about 40 years of age. Given the diffuse nature of amyloid fibril deposition, FAP is associated with various symptoms, many of which are non-specific. Common symptoms include pain, paresthesia, muscular weakness and autonomic dysfunction. Neuropathy may be accompanied by various combinations of cardiac, gastrointestinal, renal or ocular symptom. At the earliest stages, cold and pinprick sensitivity may be present. In its terminal state, the kidneys and the heart are affected.

FAP is characterized by the systemic deposition of amyloidogenic variants of the transthyretin protein, especially in the peripheral nervous system. Amyloid deposits are distributed diffusely in the peripheral nervous system, involving nerve trunks, plexuses, and sensory and autonomic ganglia. Amyloid deposits in peripheral nerves occur especially in the endoneurium, where they appear close to Schwann cells (SCs) and collagen fibrils. In severely affected nerves, endoneurial contents are replaced by amyloid, and few nerve fibers retain viability. The central nervous system (CNS) is relatively unaffected, with the exception of the ependymal lining and leptomeninges. Outside the nervous system, extensive amyloid deposits have been observed throughout connective tissue in a perivascular distribution. The receptor for advanced glycation end products (RAGE) displays increased expression in FAP tissues.

A replacement of valine by methionine at position 30 (TTR V30M) is the mutation most commonly found in FAP, although more than 100 amyloidogenic point mutations have been identified worldwide. In a particular embodiment, the subject has a genetic mutation in the TTR gene selected from Val30Met, Val50Met, Ala25Ser, Val30Leu, Phe33Val, Asp38Ala, Glu42Gly, Phe44Ser, Gly47Arg, Gly47Val, Thr49Ile, Thr49Ala, Ser50Arg, Glu54Lys, Leu55Pro, Glu61Lys, Va171Ala, Ser77Tyr, Ala97Gly, Ala109Ser, Va128Ser, Va128Met, Ala36Pro, Ile84Asn, His88Arg, Ala120Ser, Leu58Arg, Tyr69Ile, Ile107Val, Tyr114His, Ala120Ser or Ala120Thr.

A typical work-up of polyneuropathy usually includes a complete medical history, a detailed clinical neurological examination, nerve conduction studies, and routine laboratory tests. In one embodiment, the subject is diagnosed by determining a neurological impairment score (NIS) for cranial nerves, thorax, and both upper and lower limbs (0-244 points) (Dyck, P J et al. (1995) i Neurology, 45, 1115-1121). In another embodiment, the subject is diagnosed by determining a neurological disability score (NDS)(1-10 points)(Dyck, PJ, et al., (1988) Muscle and Nerve, 11, 21-32). In another embodiment, the subject is diagnosed by quantitative sensory testing (QST)(Rolke et al.(2006) Pain, 125, 197). In a further embodiment, the subject is diagnosed by autonomic function testing (AFT), such as sympathetic skin response (SSR) and the heart rate variability (HRV)(Haegele-Link et al., (2008) The Open Neurology Journal, 2, 12-19). Congo red staining may be used to histologically identify amyloid deposition. Histological identification of amyloid deposition may involve abdominal fat aspirate Once amyloid deposition is identified, immunohistochemical staining may be used to identify TTR. In certain embodiments, the subject is diagnosed by combining one of more of these methods.

In a particular embodiment, the invention provides a method of preventing neuropathic TTR-associated amyloidosis comprising administering a prophylactically effective amount of the composition disclosed herein to a subject in need thereof, thereby preventing the neuropathic TTR-associated amyloidosis.

In a particular embodiment, the invention provides a method of treating neuropathic TTR-associated amyloidosis comprising administering a therapeutically effective amount of the composition disclosed herein to a subject in need thereof, thereby treating the neuropathic TTR-associated amyloidosis.

In one embodiment, the invention provides a method of treating neuropathic TTR-associated amyloidosis comprising co-administration of an effective amount of at least one TTR kinetic stabilizer (e.g., tolcapone) and an effective amount of at least one RNAi molecule (e.g., siRNA), wherein the form of co-administration is selected from the group consisting of simultaneous administration, sequential administration, overlapping administration, interval administration, continuous administration, or a combination thereof.

In one embodiment, treatment produces a reduction in one or more clinical measures of neuropathic TTR-associated amyloidosis (as compared to the same clinical measures pre-treatment), wherein the clinical measure is selected from selected from TTR gene expression, serum TTR protein levels and/or fibril formation. The reduction in the relevant clinical measure can be measured using any suitable method including, but not limited to, the methods disclosed herein.

In exemplary embodiments, treatment produces a reduction or elimination of symptoms associated with the subject's neuropathic TTR-associated amyloidosis. Representative, non-limiting symptoms that may be reduced by the method disclosed herein include peripheral neuropathy, sexual impotence, diarrhea, constipation, urinary problems, orthostatic hypotension, cardiac problems, kidney problems, eye problems and carpal tunnel syndrome.

In one embodiment, treatment produces a reduction in a human subject's Neuropathy Impairment Score (NIS), which that measures weakness, sensation, and reflexes, especially with respect to peripheral neuropathy. (Dyck, P. et al., Neurology 1997. 49(1): pgs. 229-239). In certain embodiments, the subject's NIS score is reduced following treatment by at least about 5%, at least about 10%, at least about 15%, at least about 20%, at least about 25%, at least about 30%, at least about 35%, at least about 40%, at least about 45% or at least about 50% or more. In other embodiments, the method arrests an increasing NIS score, e.g., the method results in an about a 0% increase of the NIS score.

In a particular embodiment, treatment permits an increase in life expectancy of a subject diagnosed with FAP. In a particular embodiment, a group of FAP patients is treated and disease progression is greater than about 3 months, greater than about 6 months, greater than about 1 year, greater than about 3 years, or greater than about five years—in each case compared to patients not treated according to the method described herein.

In one embodiment, cardiac TTR-associated amyloidosis is familial amyloid cardiomyopathy (FAC) (also called hereditary ATTR-cardiomyopathy, abbreviated also as hATTR-cardiomyopathy). Infiltration of the heart from insoluble protein deposits in amyloidosis often results in restrictive cardiomyopathy that manifests late in its course with heart failure and conduction abnormalities. Subjects with cardiac amyloidosis may have an abnormal heartbeat (arrhythmia), an enlarged heart (cardiomegaly), or orthostatic hypertension.

An isoleucine 122 gene mutation of the TTR gene causes a hereditary amyloidosis primarily involving the heart without neurologic symptom, with an age of presentation typically ≥60 years and associated with African/Caribbean ethnicity. A T60A mutation is also associated with hATTR-cardiomyopathy, with an age presentation typically ≥60 years and associated with Caucasian (Irish) ethnicity. In a particular embodiment, the subject has a genetic mutation in the TTR gene selected from Asp18Glu, Ala36Asp, Ala45Asp, Ser50Ile, Thr59Arg, Thr60Ala, Glu89Lys, Gln92Lys, Val94Gly, Asp38Ala, Ser50Arg, Val122Ile, Glu89Gln, Pro24Ser or Val30Leu.

Methods of diagnosing hATTR-cardiomyopathy on a non-genetic basis include the histochemical tests described herein, as well as echocardiography with strain imaging, cardiac magnetic resonance (CMR), electrocardiography (ECG), and serum biomarker testing, including B-type natriuretic peptide (BNP or N-terminal pro-BNP) and cardiac troponin (T or I).

In one embodiment, the invention provides a method of treating hATTR-cardiomyopathy comprising administering a therapeutically effective amount of the composition disclosed herein to a subject in need thereof, thereby treating the hATTR-cardiomyopathy.

In another embodiment, the invention provides a method of treating hATTR-cardiomyopathy comprising co-administration of an effective amount of at least one TTR kinetic stabilizer (e.g., tolcapone) and an effective amount of at least one RNAi molecule (e.g., siRNA) wherein the form of co-administration is selected from the group consisting of simultaneous administration, sequential administration, overlapping administration, interval administration, continuous administration, or a combination thereof.

In one embodiment, treatment produces a reduction in one or more clinical measures of hATTR-cardiomyopathy (as compared to pre-treatment), wherein the clinical measure is selected from TTR gene expression, serum TTR protein levels and/or fibril formation. The reduction in the relevant clinical measure can be measured using any suitable method including, but not limited to, the methods disclosed herein.

In one embodiment, treatment produces a reduction or elimination of symptoms associated with the subject's hATTR-cardiomyopathy. Symptoms that may be reduced by treatment including abnormal heartbeat (arrhythmia), an enlarged heart (cardiomegaly) or orthostatic hypertension.

In one embodiment, cardiac TTR-associated amyloidosis is senile systemic amyloidosis (SSA) (also called wild-type ATTR-cardiomyopathy, abbreviated also as wtATTR-cardiomyopathy). Infiltration of the heart from insoluble protein deposits in amyloidosis often results in restrictive cardiomyopathy that manifests late in its course with heart failure and conduction abnormalities. Subjects with cardiac amyloidosis may have an abnormal heartbeat (arrhythmia), an enlarged heart (cardiomegaly), or orthostatic hypertension.

Methods of diagnosing cardiac amyloidosis include the histochemical tests described herein, as well as echocardiography with strain imaging, cardiac magnetic resonance (CMR), electrocardiography (ECG), and serum biomarker testing, including B-type natriuretic peptide (BNP or N-terminal pro-BNP) and cardiac troponin (T or I).

In one embodiment, the invention provides a method of treating wild-type ATTR-cardiomyopathy comprising administering a therapeutically effective amount of the composition disclosed herein to a subject in need thereof, thereby treating the wtATTR-cardiomyopathy.

In another embodiment, the invention provides a method of treating wtATTR-cardiomyopathy comprising co-administration of an effective amount of at least one TTR kinetic stabilizer (e.g., tolcapone) and an effective amount of at least one RNAi molecule (e.g., siRNA) wherein the form of co-administration is selected from the group consisting of simultaneous administration, sequential administration, overlapping administration, interval administration, continuous administration, or a combination thereof.

In one embodiment, treatment produces a reduction in one or more clinical measures of wtATTR-cardiomyopathy (as compared to pre-treatment), wherein the clinical measure is selected from TTR gene expression, serum TTR protein levels and/or fibril formation. The reduction in the relevant clinical measure can be measured using any suitable method including, but not limited to, the methods disclosed herein.

In one embodiment, treatment produces a reduction or elimination of symptoms associated with the subject's wtATTR-cardiomyopathy. Symptoms that may be reduced by treatment including abnormal heartbeat (arrhythmia), an enlarged heart (cardiomegaly) or orthostatic hypertension.

Leptomeningeal TTR-associated amyloidosis (hATTR-leptomeningeal) primarily affects the central nervous system. buildup of protein in leptomeninges can cause stroke and bleeding in the brain, an accumulation of fluid in the brain (hydrocephalus), difficulty coordinating movements (ataxia), muscle stiffness and weakness (spastic paralysis), seizures, and loss of intellectual function (dementia). Eye problems similar to those in the neuropathic form may also occur.

In one embodiment, the invention provides a method of treating leptomeningeal TTR-associated amyloidosis comprising administering a therapeutically effective amount of the composition disclosed herein to a subject in need thereof, thereby treating the leptomeningeal TTR-associated amyloidosis.

In one embodiment, the invention provides a method of treating leptomeningeal TTR-associated amyloidosis comprising co-administration of an effective amount of at least one TTR kinetic stabilizer (e.g., tolcapone) and an effective amount of at least one RNAi molecule (e.g., siRNA) wherein the form of co-administration is selected from the group consisting of simultaneous administration, sequential administration, overlapping administration, interval administration, continuous administration, or a combination thereof.

In exemplary embodiments, treatment produces a reduction in one or more measures of leptomeningeal TTR-associated amyloidosis (as compared to pre-treatment), wherein the clinical measure is selected from TTR gene expression, serum TTR protein levels and/or fibril formation. The reduction in the relevant clinical measure can be measured using any suitable method including, but not limited to, the methods disclosed herein.

In one embodiment, treatment produces a reduction or elimination of symptoms associated with the subject's leptomeningeal TTR-associated amyloidosis. Representative, non-limiting symptoms include stroke, bleeding in the brain, an accumulation of fluid in the brain (hydrocephalus), difficulty coordinating movements (ataxia), muscle stiffness and weakness (spastic paralysis), seizures, loss of intellectual function (dementia) and eye problems (i.e., oculoleptomeningeal issues).

In one embodiment, patients with TTR-associated amyloidosis may have one or more of a combination of symptoms associated with ATTR-cardiomyopathy, ATTR-polyneuropathy, or ATTR-leptomeningeal.

In one embodiment, treatment produces a reduction or elimination of one or more symptoms associated with the subject's ATTR-cardiomyopathy, ATTR-polyneuropathy, or ATTR-leptomeningeal.

In one embodiment, the invention provides a method of treating TTR-associated amyloidosis comprising co-administration of an effective amount of at least one TTR kinetic stabilizer (e.g., tolcapone) and an effective amount of at least one RNAi molecule (e.g., siRNA) wherein the form of co-administration is selected from the group consisting of simultaneous administration, sequential administration, overlapping administration, interval administration, continuous administration, or a combination thereof.

In one embodiment, the invention provides a method for inhibiting the expression of the TTR gene as compared a control, comprising administering the composition disclosed herein to a system (e.g., cell-free in vitro system), cell, tissue or organism.

Inhibition of TTR gene expression can be measured by any suitable method, such as measuring mRNA levels or TTR protein levels. Representative, non-limiting methods of measuring TTR gene expression include quantitative polymerase chain reaction (qPCR) amplification, RNA solution hybridization, nuclease protection, northern hybridization, gene expression monitoring with a microarray, antibody binding, radioimmunoassay, and fluorescence activated cell analysis.

In exemplary embodiments, the expression of the TTR gene is inhibited by at least about 5%, about 10%, about 15%, about 20%, about 25%, about 30%, about 35%, about 40%, about 45% or about 50% or more. In exemplary embodiments, the expression of the TTR gene is inhibited by at least about 60%, about 70% or 80% or more. In exemplary embodiments, the expression of the TTR gene is inhibited by at least about 85%, about 90% or about 95% or more.

In exemplary embodiments, the expression of the TTR gene is inhibited synergistically compared to the inhibition of the TTR gene by a TTR kinetic stabilizer (e.g., tolcapone) or a TTR genetic silencer (e.g., siRNA) when administered alone, i.e., not in combination. In exemplary embodiments, the synergism is about 1.1, about 1.2, about 1.3, about 1.4, about 1.5, about 1.6, about 1.7, about 1.8, about 1.9 or about 2.0 or more.

In one embodiment, the invention provides a method for reducing the serum TTR expression of TTR protein as compared to a control, comprising administering the composition disclosed herein to a system (e.g., cell-free in vitro system), cell, tissue or organism.

The serum TTR protein concentration can be determined directly using any methods known to one of skill in the art, e.g., an antibody based assay or an ELISA.

In exemplary embodiments, the serum TTR protein concentration is reduced by about 5%, about 10%, about 15%, about 20%, about 25%, about 30%, about 35%, about 40%, about 45% or about 50% or more. In exemplary embodiments, the expression of the TTR gene is inhibited by at least about 60%, about 70% or 80% or more. In exemplary embodiments, the expression of the TTR gene is inhibited by at least about 85%, about 90% or about 95% or more.

In exemplary embodiments, the serum TTR protein concentration is reduced synergistically compared to reduction of serum TTR protein concentration by a TTR kinetic stabilizer (e.g., tolcapone) or a TTR genetic silencer (e.g., siRNA) administered alone, i.e., not in combination. In exemplary embodiments, the synergism is about 1.1, about 1.2, about 1.3, about 1.4, about 1.5, about 1.6, about 1.7, about 1.8, about 1.9 or about 2.0 or more.

In exemplary embodiments, the serum TTR protein concentration is reduced to below about 50 ng/mL, about 45 ng/ml, about 40 ng/mL, about 35 ng/ml, about 30 ng/ml, about 25 ng/ml, about 20 ng/ml, about 15 ng/ml, about 10 ng/ml or about 5 ng/ml.

In some embodiments, the concentration of serum TTR protein is reduced to below 50 μg/ml, or to below 40 μg/ml, 25 μg/ml, or 10 μg/ml. In some embodiments, the concentration of serum TTR protein is reduced by 80%, or by 81%, 82%, 83%, 84%, 85%, 86%, 87%, 88%, 89%, 90%, or by 95%.

In one embodiment, the invention provides a method for stabilizing TTR as compared to a control, comprising administering the composition disclosed herein to a system (e.g., cell-free in vitro system), cell, tissue or organism.

Stability of the TTR protein can be determined by any suitable method, e.g., a decrease the rate of urea mediated TTR dissociation.

In one embodiment, the TTR protein is stabilized by about 2.0 kcal/mole or greater.

In one embodiment, the invention provides a method for reducing fibril formation as compared to a control, comprising administering the composition disclosed herein to a system (e.g., cell-free in vitro system), cell, tissue or organism.

In one embodiment., the disclosed method decreases the rate of acid-mediated or MeOH mediated amyloidogenesis Fibril formation can be measured by any suitable method. Fibril formation can be measured in vitro (e.g., intra or extracellularly in cell culture) or in vivo, such as TTR found in bodily fluids, including but not restricted to blood, serum, cerebrospinal fluid, tissue and organs, including but not restricted to, the heart, the kidney, peripheral nerves, meninges, the central nervous system, the eye (including the retina and vitreous fluid), the gastrointestinal tract of a subject.

In exemplary embodiments, fibril formation is reduced by about 5%, about 10%, about 15%, about 20%, about 25%, about 30%, about 35%, about 40%, about 45% or about 50% or more. In exemplary embodiments, fibril formation is reduced by at least about 60%, about 70% or 80% or more. In exemplary embodiments, fibril formulation is reduced by at least about 85%, about 90% or about 95% or more.

In exemplary embodiments, the fibril formation is reduced synergistically compared to reduction of serum TTR protein concentration by a TTR kinetic stabilizer (e.g., tolcapone) or a TTR genetic silencer (e.g., siRNA) administered alone, i.e., not in combination. In exemplary embodiments, the synergism is about 1.1, about 1.2, about 1.3, about 1.4, about 1.5, about 1.6, about 1.7, about 1.8, about 1.9 or about 2.0 or more.

Administration may be by any conventional method or route for administration of therapeutic agents, including systemic or localized routes. In general, routes of administration contemplated include but are not necessarily limited to intranasal, intrapulmonary, intramuscular, intratracheal, subcutaneous, intradermal, topical application, intravenous, rectal, nasal, oral and other parenteral routes of administration. Routes of administration may be combined, if desired, or adjusted depending upon the agent and/or the desired effect.

In a particular embodiment, the method involves administering the composition disclosed herein to a subject in need thereof by systemic or local administration.

In another particular embodiment, the method involves co-administering (i) an effective amount of at least one TTR kinetic stabilizer (e.g., tolcapone) and (ii) an effective amount of at least one RNAi molecule to a subject in need thereof by systemic or local administration. In one embodiment, the method involves co-administering tolcapone and the RNAi molecule systemically, optionally by different routes and more particularly, administering tolcapone orally (e.g., as a table) and administering the RNAi molecule by intravenous injection.

Those of skill in the art will readily appreciate that dose levels can vary as a function of the specific agent (e.g., the specific TTR kinetic stabilizer and/or the specific TTR genetic silencer), the severity of the symptoms and the susceptibility of the subject to side effects. Preferred dosages for a given compound are readily determinable by those of skill in the art by a variety of means.

Although the dosage used will vary depending upon the clinical goal to be achieved, in one embodiment, the TTR kinetic stabilizer is administered in a fixed dose between about 5 mg and about 900 mg, between about 10 mg and about 750 mg, between about 25 mg and about 500 mg, between about 50 mg and about 250 mg, or between about 75 mg and about 150 mg. In another embodiment, the TTR kinetic stabilizer is administered in a fixed dose of about 10 mg, about 12.5 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 125 mg, about 130 mg, about 140 mg, about 150 mg, about 160 mg, about 170 mg, about 175 mg, about 180 mg, about 190 mg, 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, or about 900 mg.

In an exemplary embodiment, the TTR kinetic stabilizer is tolcapone and it is administered in a fixed dose between about 5 mg and about 500 mg, between about 10 mg and about 250 mg, between about 25 mg and about 200 mg or between about 50 mg and about 150 mg. In a particular embodiment, tolcapone is administered in a fixed dose of about 5 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg or about 150 mg or more.

Although the dosage used will vary depending on the clinical goals to be achieved, in one embodiment, the dosage of the TTR gene silencer (e.g. siRNA molecule) is selected from about 5 mg and about 900 mg, between about 10 mg and about 750 mg, between about 25 mg and about 500 mg, between about 50 mg and about 250 mg, or between about 75 mg and about 150 mg. In another embodiment, the TTR kinetic stabilizer is administered in a fix dose of about 10 mg, about 12.5 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 125 mg, about 130 mg, about 140 mg, about 150 mg, about 160 mg, about 170 mg, about 175 mg, about 180 mg, about 190 mg, 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, or about 900 mg.

Depending on the dosage it may be convenient to administer the daily dosage in several dosage units.

The dosing regimen may be the same or may vary dramatically between the TTR kinetic stabilizer and TTR gene silencer. In one embodiment, administration or co-administration is once daily. In another embodiment, administration or co-administration is twice daily. In yet another embodiment, administration or co-administration is three, four, five or six times a day. In one embodiment, administration or co-administration is once every other day.

In another embodiment, administration or co-administration is once a week, month or year. In another embodiment, administration or co-administration is twice, three or four times or more per week, month or year.

The time of administration or co-administration may vary. In one embodiment, administration or co-administration is in the morning, mid-day, noon, afternoon, evening, or midnight.

We claim:

1. A composition comprising (i) an effective amount of tolcapone; (ii) an effective amount of an RNAi molecule; and (iii) a pharmaceutical carrier, wherein the RNAi molecule is suitable for use in reducing the expression of the gene encoding transthyretin (TTR) protein.

2. A method of treating familial amyloid polyneuropathy (FAP), comprising (i) administering the composition of claim 1 to a subject in need thereof, thereby treating the familial amyloid polyneuropathy.

3. A method of treating familial amyloid polyneuropathy, comprising (i) administering to a subject in need thereof a composition comprising (i) an effective amount of tolcapone; (ii) an effective amount of an RNAi molecule and (iii) a pharmaceutical carrier , wherein the RNAi molecule is suitable for use in reducing the expression of the gene encoding transthyretin protein, thereby treating the familial amyloid polyneuropathy.
